(19) United States
(12) Patent Application Publication
(10) Pub. No.: US 2022/0223286 A1
(43) Pub. Date: Jul. 14, 2022

(54) SYSTEM, METHOD AND COMPUTER READABLE MEDIUM FOR IMPROVING SYMPTOM TREATMENT IN REGARDS TO THE PATIENT AND CAREGIVER DYAD

(71) Applicant: University of Virginia Patent Foundation, Charlottesville, VA (US)

(72) Inventors: John C. Lach, Crozet, VA (US); Virginia T. LeBaron, Charlottesville, VA (US)

(73) Assignee: University of Virginia Patent Foundation, Charlottesville, VA (US)

(21) Appl. No.: 17/615,317

(22) PCT Filed: Jun. 3, 2020
(86) PCT No.: PCT/US2020/035922

§ 371 Date: Nov. 30, 2021

Related U.S. Application Data
(60) Provisional application No. 62/858,635, filed on Jun. 7, 2019.

(51) Int. Cl. G16H 50/20; G16H 50/30; G16H 10/20; G16H 20/00; G16H 40/67; G16H 80/00

(52) U.S. Cl. CPC G16H 50/20; G16H 50/30; G16H 80/00; G16H 20/00; G16H 40/67; G16H 10/20

(57) ABSTRACT

The present disclosure relates generally to monitoring and delivering in-situ real-time personalized intervention(s) for a patient and/or caregiver. More particularly, the present disclosure relates to exchanging information among components of a smart health system with mobile devices and/or smartwatches in regards to a patient and caregiver dyad based on environmental, behavioral, physiological, and contextual data of each of a patient and caregiver.

### RELATED APPLICATIONS

The present application claims benefit of priority under 35 U.S.C § 119 (e) from U.S. Provisional Application Ser. No. 62/858,635, filed Jun. 7, 2019, entitled “System for Improving Cancer Pain Management and Related Method and Computer Readable Medium thereof”; the disclosure of which is hereby incorporated by reference herein in its entirety.

### FIELD OF INVENTION

The present disclosure relates generally to monitoring and delivering in-situ real-time personalized intervention(s) for a patient and/or caregiver. More particularly, the present disclosure relates to exchanging information among components of a smart health system with mobile devices and/or smartwatches in regards to a patient and caregiver dyad based on environmental, behavioral, physiological, and contextual data of each of a patient and caregiver.

### BACKGROUND

Pain remains a significant, pervasive problem in cancer care. The biggest fear of patients diagnosed with cancer is not always dying—it is dying in pain. Likewise, family caregivers do not necessarily fear a loved one dying—they fear watching them suffer. Unfortunately, for the over 15 million Americans coping with cancer, these fears are justified. Despite decades of policy and practice efforts, along with imperatives issued by the World Health Organization, the National Academies of Medicine, the American Society of Clinical Oncology and The National Institutes of Health to improve pain management, an estimated 40-90% of patients with cancer, including those with advanced, late-stage disease—who ideally are receiving quality end of life care—still experience moderate to severe pain. Even patients with cancer enrolled in home hospice programs, which are uniquely designed to provide comprehensive support at the end of life, experience poorly managed symptoms; one study found that over 50% of hospice patients experience moderate to severe pain in the last week of life. Other research supports these findings. For example, a systematic review of the prevalence of signs and symptoms in the last 2 weeks of life, found that pain was the second highest reported symptom (52.4%) after terminal dyspnea (56.7%); relatedly, a longitudinal cohort study of adult decedents (including patients with terminal cancer) found that proxy (bereaved caregiver) reports of moderate or severe pain increased for all decedents by 20.9% between 1998 and 2010. Poorly managed cancer pain has serious ramifications, negatively affecting sleep, adherence to treatment, mood and overall quality of life—for both patients and their caregivers. Witnessing untreated pain is also a significant stressor for family caregivers, and can have a lasting, damaging psychological impact. Breakthrough cancer pain—pain that increases unpredictably above baseline pain—can be particularly difficult to manage. Breakthrough pain that escalates without adequate, prompt treatment can cause significant patient and caregiver distress, as well as unplanned healthcare utilization/emergency department visits, which may not be compatible with patient goals at end of life. Recent studies have estimated that between 25-55% of emergency department visits for patients with advanced cancer are avoidable, cost healthcare systems almost a million dollars annually, and are a major reason that patients dis-enroll from hospice programs. Effectively managing pain is a foundational element of quality cancer care, and pain is a leading reason patients with cancer are referred to palliative care, a specialty that focuses on providing holistic symptom management in the context of serious illness. Many have argued that managing pain in the context of serious illness is not only medically appropriate, but a basic human right and matter of social justice. Pain remains a significant, pervasive problem in non-cancer care as well.

Most cancer symptom management occurs in the home setting, and when patients with cancer are weakened by the effects of treatment or progression of disease, it is family caregivers who commonly assume primary responsibility for managing complex symptoms. For example, family caregivers must be able to detect and interpret physiological, social, and emotional cues to help determine the degree of pain the patient is experiencing, make independent decisions about when, and how, to intervene, and then accurately evaluate and relay to providers how well the intervention worked. These challenges can be particularly acute in the context of hospice care, where: 1) over 50% of patients receive care at home; 2) family caregivers are highly engaged in managing complex symptoms; and 3) healthcare providers (HCPs) must often coordinate care remotely. In fact, among all home care tasks, pain management is consistently rated as one of the most difficult, and stressful, by family caregivers. Complicating cancer pain management is the reality that opioids, a main-stay class of medications used to treat advanced cancer pain, are also potentially drugs of misuse. Given concerns regarding the national ‘opioid epidemic’ it is imperative that patients with cancer and family caregivers, especially those who are geographically isolated, have the support they need to safely assess and manage pain. We also know that there is a dyadic (reciprocal) and dynamic dimension to patient and caregiver distress; however, a better understanding of these relationships are essential to inform effective interventions, especially regarding pain management.

There is a long felt need for developing and optimizing personalized and effective cancer-related and other disease-related symptom management interventions.

There is a long felt need for delivering in-situ real-time personalized intervention(s) to a patient and caregiver coping with cancer or non-cancer pain management or other cancer-related or disease-related symptoms.

### SUMMARY OF ASPECTS OF EXEMPLARY EMBODIMENTS OF THE INVENTION

Smart health technology can support patients and family caregivers in managing complex, advanced symptoms. Mobile and wireless technology (collectively referred to in this disclosure as ‘smart health’), is increasingly recognized as an important tool to support personalized cancer care. Smart health has been shown to improve health outcomes for patients with a myriad of health conditions, including cancer. A benefit of smart health is the ability to collect a wide range of relevant data passively, minimizing invasiveness and burden—an important consideration for patients and family caregivers coping with the stressors of advanced cancer. Leveraging smart health technology is a critical next step to understand and characterize the symptom experience and optimally and holistically support patients and family caregivers. To date, technology-based interventions for patients with cancer have largely focused on recording and tracking self-reported symptom data and communicating the results to healthcare providers. The present inventor submits that a key gap, and research opportunity, is leveraging smart health's unique capabilities to comprehensively document the dynamic, evolving nature of complex symptoms, such as advanced cancer pain, in real-time. This gap is critical to address, as it is foundational to developing personalized and effective symptom management interventions. Pain management is too often a one-size-fits-all approach. While specific medications or strategies may be prescribed, the nuances of how—for this particular patient and caregiver—to mitigate pain from escalating, exactly when to initiate a pain alleviating strategy, or the most effective ways to modify the environment to reduce pain, are typically discovered through trial and error, requiring a luxury of time not afforded to those with advanced cancer.

In sum, the present inventor submits that to truly deliver personalized strategies for cancer pain management, then we must first understand the personalized experience of cancer pain. The present inventor hypothesizes that individuals, and dyads, will display a unique ‘digital fingerprint’ (or phenotype) of the advanced cancer pain experience—that if better understood can be utilized to inform and deliver personalized, timely pain alleviating interventions.

An aspect of an embodiment shall deploy a novel, smart health system, Behavioral and Environmental Sensing and Intervention for Cancer (BESI-C), to comprehensively characterize the complexity of the advanced cancer pain experience in the home setting. Briefly, for example in an embodiment, BESI-C is a package of wearable (smartwatch) and home-based sensors designed to unobtrusively and reliably collect behavioral, environmental, physiological, and contextual data. The sensing components of BESI-C collect heterogeneous passive and active data streams that are integrated to paint an in-depth picture about the experience of cancer pain from the perspective of both an individual and dyad. In essence, when a patient or caregiver records a pain event on their respective smartwatch, BESI-C provides a comprehensive ‘snap shot’ of exactly what is occurring at and around the time of the event. These data can then inform and train personalized models that find relations between behavioral, environmental, physiological, and contextual factors and pain events and inform real-time notifications for early intervention. The present inventor's research and technique addresses critical gaps, as it will leverage innovative smart health technology to, among other things: 1) capture the complex experience of advanced cancer pain in the home setting from a dyadic perspective (e.g., Aim 1); 2) consider the role of multiple factors on the pain experience, including environmental, behavioral, physiological, and contextual factors (e.g., Aims 1,2,3); 3) explore how to best communicate shared data with key stakeholders (e.g., Aim 2); 4) discover predictors of breakthrough (acute) pain events (e.g., Aim 3); and 5) inform ways to support family caregivers to manage distressing symptoms, especially pain, in the home environment in real-time (e.g., Aims 1,2,3). Successful completion of the aims will advance scientific knowledge by proposing a paradigm shift in how we manage advanced symptoms in the home setting and how we deliver timely and tailored symptom relief that addresses the needs of both patients and family caregivers. Importantly, for example, an embodiment of the present invention system and method provides a scalable and reproducible model to decrease disparities in pain management and improve access to palliative care services.

An aspect of an embodiment of the present invention provides a system, method and computer readable medium for, among other things, monitoring and delivering in-situ real-time personalized intervention for a patient coping with cancer or non-cancer pain management or cancer-related or other disease-related symptoms regarding a patient and caregiver dyad. Patient and caregiver dyadic in-situ data is collected which may include environmental data, behavioral data, physiological data, and contextual data of each of the patient and caregiver. Also, cancer or non-cancer pain events data or cancer-related or other disease-related symptom events data of the patient is collected. Next, the relationship of the patient and caregiver dyadic in-situ data to the cancer or non-cancer pain events data or cancer-related or other disease-related symptom events data of patient is determined. As a result, real-time personalized intervention information of the patient and/or caregiver, based on the determined relationship, can be generated and communicated for appropriate action to be undertaken by the caregiver, patient, both the caregiver and patient, and/or health care provider, as well as to cloud services and electronic health records (EHR) and so forth.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwired or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal (such as a horse in a veterinarian, farm, or equestrian setting, etc.). It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g. rat, dog, pig, monkey), etc. It should be appreciated that the subject may be any applicable human patient, for example.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 is a block diagram depicting a system for monitoring and delivering in-situ real-time personalized interventions to mobile user devices, in accordance with certain example embodiments.

FIG. 2 is a block diagram depicting a computing machine and a module, in accordance with certain example embodiments.

FIG. 3 provides a screenshot of an overall study design, in accordance with certain example embodiments.

FIG. 4 provides a screenshot of an overview of an embodiment of a BESI-C system architecture, in accordance with certain example embodiments.

FIG. 5 provides a screenshot of selected examples of BESI-C smartwatch screen displays, in accordance with certain example embodiments.

FIG. 6 provides a screenshot of BESI-C assessment model, in accordance with certain example embodiments.

FIG. 7 provides a screenshot of selected examples of “BESI-C Application” screen displays, in accordance with certain example embodiments.

FIG. 8 provides a screenshot of selected examples of “Patient Pain EMA” pertaining to, for example but not limited thereto, how a patient initially marks and describes a pain event, in accordance with certain example embodiments.

FIG. 9 provides a screenshot of selected examples of “Patient Follow-up EMA” pertaining to, for example but not limited thereto, how a patient describes pain 30 minutes after using a pharmacological or non-pharmacological strategy to reduce pain, in accordance with certain example embodiments.

FIG. 10 provides a screenshot of selected examples of “Patient Manual End of Day EMA” pertaining to, for example but not limited thereto, a patient survey at the end of the day that assesses general patient well-being, contextual factors and behaviors, in accordance with certain example embodiments.

FIG. 11 provides a screenshot of selected examples of “Patient Automatic End of Day EMA” pertaining to, for example but not limited thereto, a patient survey at the end of the day that assesses general patient well-being, contextual factors and behaviors, in accordance with certain example embodiments.

FIG. 12 provides a screenshot of selected examples of “Caregiver Pain EMA” pertaining to, for example but not limited thereto, how a caregiver initially marks and describes their perspective of a patient pain event, in accordance with certain example embodiments.

FIG. 13 provides a screenshot of selected examples of “Caregiver Follow-up EMA” pertaining to, for example but not limited thereto, how a caregiver describes pain 30 minutes after they report a patient uses a pharmacological or non-pharmacological strategy to reduce pain, in accordance with certain example embodiments.

FIG. 14 provides a screenshot of selected examples of “Caregiver Manual End of Day EMA” pertaining to, for example but not limited thereto, a caregiver survey at the end of the day that assesses general caregiver well-being, contextual factors and behaviors, in accordance with certain example embodiments.

FIG. 15 provides a screenshot of selected examples of “Caregiver Automatic End of Day EMA” pertaining to, for example but not limited thereto, a caregiver survey at the end of the day that assesses general caregiver well-being, contextual factors and behaviors, in accordance with certain example embodiments.

### DETAILED DESCRIPTION OF EXEMPLARY EMBODIMENTS

An aspect of an embodiment of the present invention provides a computer-implemented method to deliver in-situ real-time personalized intervention(s) to a patient and/or caregiver coping with cancer or non-cancer pain management or cancer-related or other disease-related symptoms. A patient user can employ a patient user device (such as a smartwatch, mobile device, smartphone, or personal digital assistant (PDA)) that may be configured to collect/gather and/or receive passive and active data and communicate information and transmit data. A caregiver can employ a caregiver user device (such as a smartwatch, mobile device, smartphone or personal digital assistant (PDA)) that may be configured to collect/gather and/or receive passive and active data and communicate information and transmit data. Other supplemental sensors and detectors can be employed to collect/gather and/or receive passive and active data as well as transmit data. The computer implement method and system offers, among other things, a novel approach to deliver personalized symptom management strategies to improve patient and caregiver outcomes and reduce disparities in pain management or cancer-related symptoms.

In an embodiment of the system, a patient user and caregiver user opens a patient user interface on the patient user device (such as a smartwatch or mobile phone) and caregiver user interface on the caregiver user device (such as a smartwatch or mobile phone), respectively, to collect active data that requires participant (patient and caregiver) to directly interface with the system by answering questions or marking an event. Moreover, passive data may be collected without user effort on the patient user device and caregiver user device, as well as other devices and components of the system. The data may be related to cancer or non-cancer pain or cancer-related or other disease-related symptoms from both the patient and caregiver perspective using a cyber-physical platform comprised of wearable devices (e.g., smartwatches), in-situ sensors and networks, and secure cloud services.

In an embodiment, in-situ sensor and devices are provided to obtain various environmental data, behavioral data, physiological data, and contextual data of each of the patient and caregiver.

In an embodiment, the system and method may include a computer, processor, computer network, or computer server (any of which may include cloud platform or cloud services) may be provided to collect cancer or non-cancer pain events data or cancer-related or other disease-related symptom events data of the patient.

In an embodiment, the system and method may include a computer, processor, computer network, or computer server (any of which may include cloud platform or cloud services) may be provided to relate the patient and caregiver dyadic in-situ data to the cancer or non-cancer pain events data or cancer-related or other disease-related symptom events data of patient. The relation may be accomplished with predictive pain algorithms. These data can then inform and train personalized models that find relations between behavioral, environmental, physiological, and contextual factors and pain events and inform real-time notifications for early intervention. For example, the number of pain events will be compared between patients using mixed effects Poisson regression (which can account for potentially variable amount of data collection time and look for similarities across dyads), and to test for differences in rates by demographic, clinical and environmental characteristics. Sensor data will be summarized over time using standard measures, such as mean, variance, max, and/or min, for inclusion in the regression models. Structure equation models may be used to explore associations and develop hypotheses between variables that have recursive relationships.

In an embodiment, the system and method may include a computer, processor, computer network, or computer server (any of which may include cloud platform or cloud services) and may be provided to generate real-time personalized intervention information of the patient and/or caregiver, based on relations, such as train personalized models that find relations (and for example, correlations) between behavioral, environmental, physiological, and contextual factors and pain events and inform real-time notifications for early intervention.

In an embodiment, the system and method may include a computer, processor, computer network, or computer server (any of which may include cloud platform or cloud services) and may be provided to communicate collected data and/or the real-time personalized intervention information, for appropriate action to be undertaken, to anyone or more of the following: the caregiver; both the caregiver and patient, the patient, health care provider, other designated individuals/agencies approved by the user, data storage device, output device, network server, computer processor device, cloud services, electronic health record (EHR), or display device.

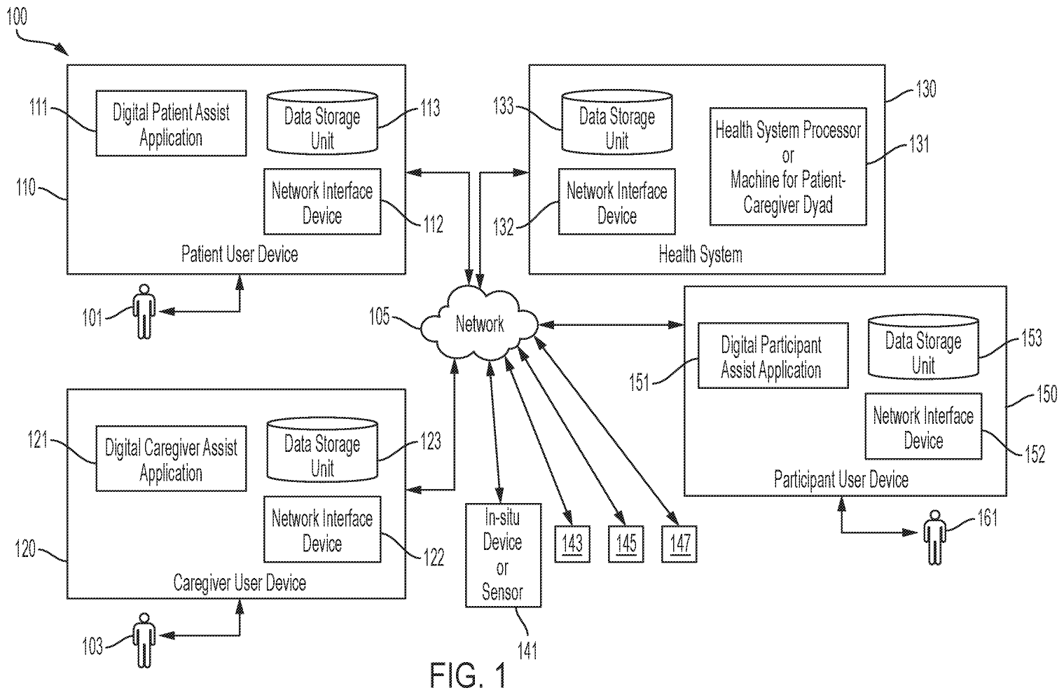

FIG. 1 is a block diagram depicting a system for delivering in-situ real-time personalized intervention for a patient coping with cancer or non-cancer pain management or other cancer-related or other disease-related symptoms with a mobile user device such as a smartwatch or smartphone, in accordance with certain example embodiments. As depicted in FIG. 1, the system 100 includes network devices 110, 120 and 130 (as well as other network devices 141, 143, 145, 147 and 150) that are configured to communicate with one another via one or more networks 105.

Each network 105 includes a wired or wireless telecommunication means by which network devices (including devices 110, 120 and 130, as well as network devices 141, 143, 145, 147 and 150) can exchange data. For example, each network 105 can include a local area network (“LAN”), a wide area network (“WAN”), an intranet, an Internet, a mobile telephone network, or any combination thereof. Throughout the discussion of example embodiments, it should be understood that the terms “data” and “information” are used interchangeably herein to refer to text, images, audio, video, or any other form of information that can exist in a computer-based environment.

Each network device 110, 120 and 130, as well as network devices 141, 143, 145, 147 and 150) includes a device having a communication module capable of transmitting and receiving data over the network 105. For example, each network device 110, 120 and 130 can include a smartwatch (or other wearable or stationary computer or processor-based device), server, desktop computer, laptop computer, tablet computer, mobile device, smartphone, handheld computer, personal digital assistant (“PDA”), or any other wired or wireless, processor-driven device. In the example embodiment depicted in FIG. 1, the network devices 110, 120 and 130 are operated in association with patient-user 101, caregiver-user 103, or other operator. It is to be understood that a participant user device 150 may be operated by a healthcare provider or clinician 161 with the other network devices.

In an embodiment, the patient user 101 and caregiver user 103 can use a network interface device 112 and 122, such as a web browser application or a stand-alone application (or other transfer protocols such as frame relay, IP, TCP, UDP, HTTP, etc.), to view, download, upload, report, or otherwise access surveys and intervention, documents or web pages via a distributed network 105. The network 105 includes a wired or wireless telecommunication system or device by which network devices (including devices 110, 120, 130, and 150) can exchange data. For example, the network 105 can include a local area network (“LAN”), a wide area network (“WAN”), an intranet, an Internet, storage area network (SAN), personal area network (PAN), a metropolitan area network (MAN), a wireless local area network (WLAN), a virtual private network (VPN), a cellular or other mobile communication network, Bluetooth, NFC, or any combination thereof or any other appropriate architecture or system that facilitates the communication of signals, data, and/or messages. Throughout the discussion of example embodiments, it should be understood that the terms “data” and “information” are used interchangeably herein to refer to text, images, audio, video, or any other form of information that can exist in a computer based environment.

In an embodiment, the network interface device 112 and 122 of the patient user device 110 and caregiver user device 120, respectively, can communicate with the Health system 130 server via its network interface device 132 through a web server, or other computer can that establish a communication via near field communication (“NFC”), BLUETOOTH, Wi-Fi, infrared, or any other suitable communication technology. In an embodiment, the health system 130 (e.g., patient-caregiver dyad health system) may have a computer processor or machine 131 available for the patient and caregiver dyad dynamics.

In an embodiment, the patient user device 110 and caregiver user device 120 may include a digital patient assist application 111 and digital caregiver assist application 121, respectively. The digital patient assist application 111 and digital caregiver assist application 121 may encompass any application, hardware, software, or process the user devices 110 and 120, respectively, may employ to assist the patient user 101 and caregiver user 103 in completing a survey or receiving personalized intervention.

In an embodiment, in-situ sensor or devices 141, 143, 145, and 147 may be in communication with the network 105. The in-situ sensor or devices 141, 142, 145, and 147 may be dispersed strategically in-situ such as at the patient resident setting or the given environment that which the patient is occupying, along with the caregiver.

In an embodiment, the digital patient assist application 111 and digital caregiver assist application 121 are operable to allow a patient user 101 and caregiver user 103 to configure an account, report out, download ordering information, such as a menu or survey, and interact with a health system 130 to partake in the monitoring and delivering in-situ real-time personalized intervention(s) for a patient coping with cancer or non-cancer pain management or cancer-related or other disease-related symptoms in regards to a patient and caregiver dyad. The digital patient assist application 111 and digital caregiver assist application 121 are further operable to store patient and caregiver dyadic in-situ data or information in a data storage unit 113 and 123 stored on or coupled to the patient user device 110 and caregiver user device 120.

The patient user device 110 also includes a data storage unit 113 accessible by the digital wallet patient assist application 111, and the network interface device 112. Similarly, the caregiver user device 120 also includes a data storage unit 123 accessible by the digital caregiver assist application 121, and the network interface device 122. The example data storage units 113 and 123 can include one or more tangible computer-readable storage devices. The data storage unit 113 can be stored on the patient user device 110 or can be logically coupled to the patient user device 110. For example, the data storage unit 113 can include on-board flash memory and/or one or more removable memory cards or removable flash memory. Similarly, the data storage unit 123 can be stored on the caregiver user device 120 or can be logically coupled to the caregiver user device 120. For example, the data storage unit 123 can include on-board flash memory and/or one or more removable memory cards or removable flash memory.

Alternatively, the functions of one or more of the components of the patient user device 110 and caregiver user device 120 can be performed in a cloud computing system (not pictured). For example, the data storage units 113 and 123 can be embodied by a storage device on a remote server operating as a cloud computing environment for the patient user device 110 and caregiver user device 120. In another example, some or all of the functions of the digital patient assist application 111 and the digital caregiver assist application 121 can be performed in a cloud computing environment. For example, the algorithms and calculations required during the monitoring and delivery of real-time personalized intervention information of a patient user 101 and caregiver user 120 in conjunction with a health system 130 can be performed by a remote server operating as a cloud computing environment.

The health system 130 represents an entity that provides data, algorithms, modeling, analyses, simulations, computer processing, and know-how of medical care and support of various products disclosed for the patient user 101, caregiver 103, or participant user 161 to acquire or use. The health system 130 can include a web server to host a website and an order processing application. The web server may be embodied as a server operating at the health system location or in a remote location. The web server may be the system employed by the health system 130 to operate the production, sales, operations, calculations, computer algorithmic processing, modeling, intervention, monitoring, reporting, treatment or other functions of the health system 130.

The health system 130 is configured to store and exchange information and data and perform other suitable functions. The healthcare system 130 may be an independent system or may be a third party application, or any other system that may manage and interact with the Network 105 as disclosed herein.

The healthcare system 130 can contain a data storage unit 133 that can include one or more tangible computer-readable storage devices or various machines, computers, processors, as well as make-up aspects of a server farm or cloud servers. The data storage unit 133 can be stored on the web server or can be logically coupled to the web server. For example, the data storage unit 133 can include on-board flash memory and/or one or more removable memory cards or removable flash memory.

It will be appreciated that the network connections shown are an example and other means of establishing a communications link between the computers and devices can be used. Moreover, those having ordinary skill in the art having the benefit of the present disclosure will appreciate that the health system 130 and the patient user device 110 and caregiver user device 120 illustrated in FIG. 1 can have any of several other suitable computer system configurations. For example, a patient user device 110 and caregiver user device 120 embodied as a smartwatch, mobile phone or handheld computer may not include all the components described above.

Moreover, in an embodiment, in addition to the health system 130, patient user device 110 and caregiver user device 120 as illustrated in FIG. 1 there may be a participant user device 150 for use by a healthcare provider or clinician 161. The third-party user device 150 may also include a data storage unit 153, a digital third-party assist application 151, and the network interface device 152. In an embodiment, participant user device 150 is configured to allow a participant user 161 to operate and interact with the participant user device 150, and which may be communicably coupled to the network 105. In an embodiment, any of the functionalities associated with the corresponding components in the system 100 (or operating environment), such as the patient user device 110 and caregiver user device 120 (as well as the health system 130), may be provided and achieved for the third-party user device 150. For example, the digital participant assist application 151 may encompass any application, hardware, software, or process the participant user device 150 may employ to assist the participant user 161 interact and communicate with the network 105.

In an embodiment, the digital participant assist application 151 is operable to allow a participant user 161 (e.g., healthcare provider or clinician) to configure an account, report out, download ordering information, such as a menu or survey, and interact with a health system 130 to partake in the monitoring and delivering in-situ real-time personalized intervention for a patient coping with cancer or non-cancer pain management or cancer-related or other disease-related symptoms in regards to a patient and caregiver dyad.

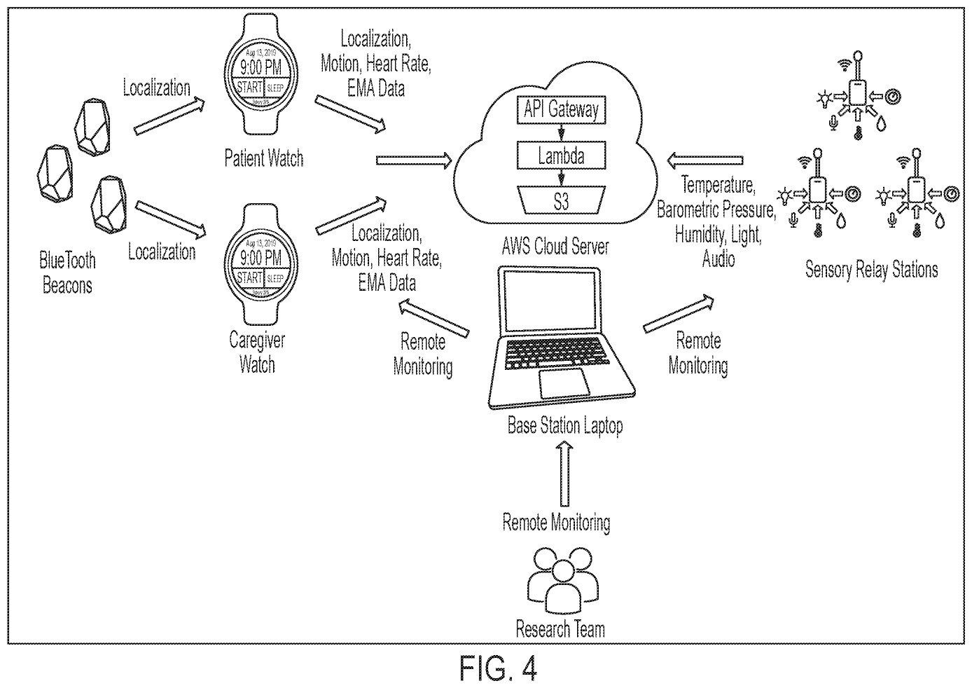

Additionally, in an embodiment, any of the functionalities associated with the components disclosed in FIGS. 4-5 and 7-15 may be implemented within the system 100 (or operating environment) reflected in FIG. 1.

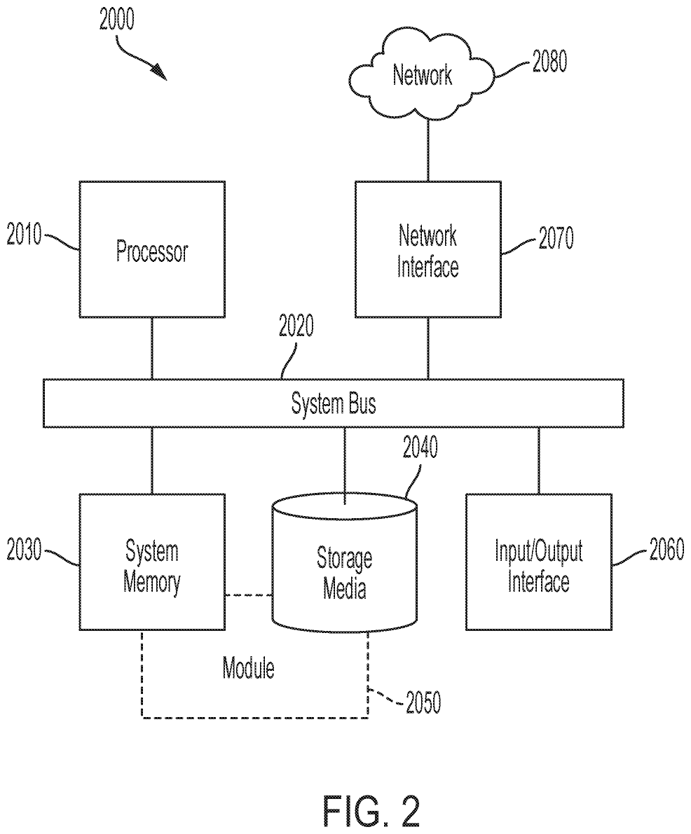

As shall be discussed below, the systems, components, and methods associated with FIGS. 2-15 may be employed in the context of the invention disclosed in components of the example operating environment 100. The example embodiments can include one or more computer programs that embody the functions described in FIGS. 2-15. However, it should be apparent that there could be many different ways of implementing aspects of the example embodiments in computer programming, and these aspects should not be construed as limited to one set of computer instructions. Further, a skilled programmer would be able to write such computer programs to implement example embodiments based on any flow charts and associated description in the application text. Therefore, disclosure of a particular set of program code instructions is not considered necessary for an adequate understanding of how to make and use the example embodiments. Further, those skilled in the art will appreciate that one or more acts described may be performed by hardware, software, or a combination thereof, as may be embodied in one or more computing systems.

### Other Example Embodiments

Users may be allowed to limit or otherwise affect the operation of the features disclosed herein. For example, users may be given opportunities to opt-in or opt-out of the collection or use of certain data or the activation of certain features. In addition, users may be given the opportunity to change the manner in which the features are employed. Instructions also may be provided to users to notify those regarding policies about the use of information, including personally identifiable information, and manners in which each user may affect such use of information. Thus, information can be used to benefit a user, if desired, through receipt of relevant notifications, offers, reports, surveys, intervention, treatment, or other information, without risking disclosure of personal information or the user's identity.

One or more aspects of the invention may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing the invention in computer programming, and the invention should not be construed as limited to any one set of computer program instructions. Further, a skilled programmer would be able to write such a computer program to implement an embodiment of the disclosed invention based on the associated description in the application text. Therefore, disclosure of a particular set of program code instructions is not considered necessary for an adequate understanding of how to make and use the invention. Further, those skilled in the art will appreciate that one or more aspects of the invention described herein may be performed by hardware, software, or a combination thereof, as may be embodied in one or more computing systems. Moreover, any reference to an act being performed by a computer should not be construed as being performed by a single computer as more than one computer may perform the act.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of the invention. Accordingly, such alternative embodiments are included in the inventions described herein.

An embodiment of the invention can be used with computer hardware and software that performs the methods and processing functions described herein. As will be appreciated by those having ordinary skill in the art, the systems, methods, and procedures described herein can be embodied in a programmable computer, computer executable software, or digital circuitry. The software can be stored on computer readable media. For example, computer readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (“FPGA”), etc.

Although specific embodiments of the invention have been described above in detail, the description is merely for purposes of illustration. Various modifications of, and equivalent blocks and components corresponding to, the disclosed aspects of the example embodiments, in addition to those described above, can be made by those having ordinary skill in the art without departing from the spirit and scope of the invention defined in the following claims, the scope of which is to be accorded the broadest interpretation so as to encompass such modifications and equivalent structures.

FIG. 2 depicts a computing machine 2000 and a module 2050 in accordance with certain example embodiments. The computing machine 2000 may correspond to any of the various computers, servers, mobile devices, smartphones, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, personal digital assistant (PDA), smartwatch, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, biometric readers, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

### Method

In an embodiment, a computer-implemented method is provided for monitoring and delivering in-situ real-time personalized intervention(s) for a patient (coping with cancer or non-cancer pain management or cancer-related or other disease-related symptoms) by exchanging information with mobile devices and/or smartwatches in regards to a patient and caregiver dyad. The method may be provided, for example but not limited thereto, in the operating environment 100 such as shown in FIG. 1. The method may comprise collecting, by one or more computer devices associated with a health system 130, patient and caregiver dyadic in-situ data, wherein the patient and caregiver dyadic in-situ data is received from a patient user computing device 110 and a caregiver user computing device 120. In an embodiment, the patient user computing device 110 and the caregiver user computing device 120 associated with the health system 130 are separate and distinct from the health system 130. In an embodiment, the patient and caregiver dyadic in-situ data may include, but is not limited thereto, the following: environmental data, behavioral data, physiological data, and contextual data of each of a patient and caregiver. In an embodiment, the method may include receiving, by one or more computer devices associated with the health system 130, cancer or non-cancer pain events data or cancer-related or other disease-related symptom events data of the patient 101 based on cancer or non-cancer pain events data or cancer-related or other disease-related symptom events data of the patient collected from the patient user computing device 110 and/or the caregiver user computing device 120. In an embodiment, the method may include storing, by one or more computer devices associated with the health system 130, the cancer or non-cancer pain events data or cancer-related or other disease-related symptom events data of the patient 101. In an embodiment, the method may include relating, by one or more computer devices associated with the health system 130, the patient and caregiver dyadic in-situ data to the cancer or non-cancer pain events data or cancer-related or other disease-related symptom events data of patient 101. In an embodiment, the method may include generating, by one or more computer devices associated with the health system 130, real-time personalized intervention information for the patient 101 and/or caregiver 103, based on the relation. In an embodiment, the method may include communicating, by one or more computer devices associated with the health system 130, the real-time personalized intervention information, to the patient user computing device 110 and caregiver user computing device 120 for appropriate action to be undertaken, to anyone or more of the following: the caregiver 103, the patient 101, or both the caregiver 103 and patient 101.

Still referring generally to the operating environment 100 of FIG. 1, in an embodiment, the method may further include communicating, by one or more computer devices associated with the health system 130, the real-time personalized intervention information, to a participant user computing device 150 for appropriate action to be undertaken. In an embodiment, the participant user computing device 150 is associated with a health care provider user 161 or a clinician user 161 (or other third-party as desired or required). In an embodiment, the participant user computing device 150 associated with the health system 130 is separate and distinct from the health system 130.

Still referring generally to the operating environment 100 of FIG. 1, in an embodiment, the real-time personalized intervention may comprise, but not limited thereto, at least one or more of any combination of the following: providing guidance of treatment for the patient and/or caregiver; predicting occurrence of cancer or non-cancer pain events and/or magnitude of cancer or non-cancer pain events of patient; or predicting cancer-related or other disease-related symptom events and/or magnitude of cancer related or other disease-related symptoms of patient 101.

Still referring generally to the operating environment 100 of FIG. 1, in an embodiment, providing guidance of treatment for the patient 101 and/or caregiver 103 includes, but not limited thereto, at least one or more of any combination of the following: providing guidance regarding dosing and timing of medication for the patient; providing guidance of pain management for the patient; providing non-pharmacological treatment for the patient; or providing behavioral, environmental or contextual modifications for the patient and/or caregiver.

Still referring generally to the operating environment 100 of FIG. 1, in an embodiment, at least one or more of the environmental data, behavioral data, physiological data, and contextual data are detected or sensed by an in-situ sensor or in-situ detector 141, 143, 145, 147.

Still referring generally to the operating environment 100 of FIG. 1, in an embodiment, the environmental data may include ambient factors, in-situ, wherein in-situ defines a patient resident setting or the like, for example. In an embodiment, the ambient factor may include, but is not limited thereto, at least one or more of the following: temperature, light, noise, humidity or barometric pressure.

Still referring generally to the operating environment 100 of FIG. 1, the behavioral data may include, but is not limited thereto, at least one or more of the following: ecological momentary assessment (EMA) data of patient and ecological momentary assessment (EMA) data of caregiver. In an embodiment, the EMA related behavioral data may include, but is not limited thereto, at least one or more of the following: behavioral factors pertaining to what the patient 101 or caregiver 103 indicates as such actions that they do or actions that they take or report to take; appetite of the patient and/or caregiver; or energy level or fatigue level of the patient and/or caregiver. In an embodiment, the EMA related behavioral data may include, but is not limited thereto, at least one or more of the following: pain medication use, reasons pain medication was not taken, or non-pharmacological strategies used to try to manage pain.

Still referring generally to the operating environment 100 of FIG. 1, in an embodiment, the physiological data may include, but is not limited thereto, at least one or more of the following: activity, movement, sleep, rest, or heart rate of the patient 101 and caregiver 103.

Still referring generally to the operating environment 100 of FIG. 1, in an embodiment, the contextual data may include, but is not limited thereto, at least one or more of the following: ecological momentary assessment (EMA) data of patient or ecological momentary assessment (EMA) data of the caregiver.

Still referring generally to the operating environment 100 of FIG. 1, in an embodiment, the EMA related contextual data may include, but is not limited thereto, at least one or more of the following: factors pertaining to what is happening around the patient 101 or caregiver 103 or factors that may influence their experience; appetite of the patient and/or caregiver; or energy level or fatigue level of the patient and/or caregiver.

Still referring generally to the operating environment 100 of FIG. 1, in an embodiment, in-situ may define a patient resident setting or the like; and the EMA related contextual data may include, but is not limited thereto, at least one or more of the following: pain severity; how busy/active was the patient resident setting; distress levels; sleep quality and quantity; mood; current location; time spent outside the patient resident setting; activity level; energy level; fatigue; appetite; room in the patient resident setting where they spent most time; how much time was spent with the other member of the dyad; time spent with other people; overall pain interference; or overall distress levels.

Still referring generally to the operating environment 100 of FIG. 1, in an embodiment, wherein in-situ being defined as a patient resident setting, and the contextual data may include, but is not limited thereto, at least one or more of the following: location of the patient 101 and caregiver 103 within the patient resident setting; or location of the patient and caregiver relative to one another, within the patient resident setting to define relative location. In an embodiment, the contextual data may further comprise: the relative location of the patient 101 and caregiver 103 when a pain event occurs.

### Computer Program Product

In an embodiment, a computer program product may be provided comprising a non-transitory computer readable storage device having computer-executable program instructions embodied thereon that when executed by a computer processes information from mobile devices and/or smartwatches for monitoring and delivering in-situ real-time personalized intervention for a patient. In an embodiment, the patient is coping with cancer or non-cancer pain management or cancer-related or other disease-related symptoms in the context of a patient and caregiver dyad. The computer-executable program instructions may be provided, for example but not limited thereto, in the operating environment 100 such as shown in FIG. 1. The computer-executable program instructions may comprise: program instructions to collect patient and caregiver dyadic in-situ data, wherein the patient and caregiver dyadic in-situ data is received from a patient user computing device 110 and a caregiver user computing device 120. In an embodiment, the patient user computing device 110 and the caregiver user computing device 120 are separate and distinct from the computer. In an embodiment, the patient and caregiver dyadic in-situ data may include, but is not limited thereto, the following: environmental data, behavioral data, physiological data, and contextual data of each of a patient and caregiver. In an embodiment, the computer-executable program instructions may include program instructions to receive cancer or non-cancer pain events data or cancer-related or other disease-related symptom events data of the patient based on pain events data or cancer-related or other disease-related symptom events data of the patient collected from the patient user computing device 110 and/or the caregiver user computing device 120. In an embodiment, the computer-executable program instructions may include program instructions to store the cancer or non-cancer pain events data or cancer-related or other disease-related symptom events data of the patient 101. In an embodiment, the computer-executable program instructions may include program instructions to relate the patient and caregiver dyadic in-situ data to the cancer or non-cancer pain events data or cancer-related or other disease-related symptom events data of patient 101. In an embodiment, the computer-executable program instructions may include program instructions to generate real-time personalized intervention information of the patient 101 and/or caregiver 103, based on the relation. In an embodiment, the computer-executable program instructions may include program instructions to communicate the real-time personalized intervention information, to the patient user computing device 110 and the caregiver user computing device 120 for appropriate action to be undertaken, to anyone or more of the following: the caregiver 103, the patient 101, or both the caregiver 103 and patient 101.

In an embodiment, the computer-executable program instructions may further include program instructions to communicate the real-time personalized intervention information, to a participant user computing device 150 for appropriate action to be undertaken. The computer-executable program instructions may be provided, for example but not limited thereto, in the operating environment 100 such as shown in FIG. 1, but not necessarily. For example, in an embodiment, the participant user computing device 150 is associated with a health care provider user 161 or a clinician user 161 (or other third-party as desired or required). In an embodiment, the participant user computing device 150 is separate and distinct from the computer.

In an embodiment, the real-time personalized intervention may comprise, but not limited thereto, at least one or more of any combination of the following: providing guidance of treatment for the patient and/or caregiver; predicting occurrence of cancer or non-cancer pain events and/or magnitude of cancer or non-cancer pain events of patient; or predicting cancer-related or other disease-related symptom events and/or magnitude of cancer related or other disease-related symptoms of patient. Still yet, in an embodiment, the providing guidance of treatment for the patient 101 and/or caregiver 103 includes at least one or more of any combination of the following: providing guidance regarding dosing and timing of medication for the patient; providing guidance of pain management for the patient; providing non-pharmacological treatment for the patient; or providing behavioral, environmental or contextual modifications for the patient and/or caregiver.

In an embodiment, at least one or more of the environmental data, behavioral data, physiological data, and contextual data are detected or sensed by an in-situ sensor or in-situ detector 141, 143, 145, 147.

### System

In an embodiment, a system may be provided to monitor and deliver in-situ real-time personalized intervention to mobile devices and/or smartwatches for a patient (e.g., coping with cancer or non-cancer pain management or cancer-related or other disease-related symptoms) in regards to a patient and caregiver dyad. The system may be provided, for example but not limited thereto, as part of the operating environment 100 such as shown in FIG. 1. In an embodiment, the system may comprise: a storage resource; a network module 105; and a processor, wherein the processor is communicatively coupled to the storage resource and the network module 105. In an embodiment, the processor executes application code instructions that are stored in the storage resource and that cause the system to: collect patient and caregiver dyadic in-situ data for a health system 130, wherein the patient and caregiver dyadic in-situ data is received from a patient user computing device 110 and a caregiver user computing device 120. In an embodiment, the patient user computing device 110 and the caregiver user computing device 120 associated with the health system 130 are separate and distinct from the processor. In an embodiment, the patient and caregiver dyadic in-situ data may include, but is not limited thereto, the following: environmental data, behavioral data, physiological data, and contextual data of each of a patient and caregiver. In an embodiment, the processor may be further configured to execute application code instructions that are stored in the storage resource and that cause the system to: receive cancer or non-cancer pain events data or cancer-related or other disease-related symptom events data of the patient based on cancer or non-cancer pain events data or cancer-related or other disease-related symptom events data of the patient collected from the patient user computing device 110 and/or the caregiver user computing device 120. In an embodiment, the processor may be further configured to execute application code instructions that are stored in the storage resource and that cause the system to: store the cancer or non-cancer pain events data or cancer-related or other disease-related symptom events data of the patient. In an embodiment, the processor may be further configured to execute application code instructions that are stored in the storage resource and that cause the system to: relate the patient and caregiver dyadic in-situ data to the cancer or non-cancer pain events data or cancer-related or other disease-related symptom events data of patient. In an embodiment, the processor may be further configured to execute application code instructions that are stored in the storage resource and that cause the system to: generate real-time personalized intervention information of the patient and/or caregiver, based on the relation. In an embodiment, the processor may be further configured to execute application code instructions that are stored in the storage resource and that cause the system to communicate the real-time personalized intervention information to the patient user computing device 110 and the caregiver user computing device 120 for appropriate action to be undertaken, to anyone or more of the following: the caregiver 103, the patient 101, or both the caregiver 103 and patient 101.

Still referring generally to the system as part of the operating environment 100 of FIG. 1, in an embodiment, the processor may be further configured to execute application code instructions that are stored in the storage resource and that cause the system to communicate the real-time personalized intervention information to a participant user computing device 150 for appropriate action to be undertaken. In an embodiment, the participant user computing device 150 is associated with a health care provider user 161 or a clinician user 161 (or other third-party as desired or required). In an embodiment, the participant user computing device 150 associated with the health system 130 is separate and distinct from the processor.

Still referring generally to the system as part of the operating environment 100 of FIG. 1, in an embodiment, the real-time personalized intervention may comprise, but not limited thereto, at least one or more of any combination of the following: providing guidance of treatment for the patient and/or caregiver; predicting occurrence of cancer or non-cancer pain events and/or magnitude of cancer or non-cancer pain events of patient; or predicting cancer-related or other disease-related symptom events and/or magnitude of cancer related or other disease-related symptoms of patient.

Still referring generally to the system as part of the operating environment 100 of FIG. 1, in an embodiment, providing guidance of treatment for the patient and/or caregiver may include, but not is limited thereto, at least one or more of any combination of the following: providing guidance regarding dosing and timing of medication for the patient; providing guidance of pain management for the patient; providing non-pharmacological treatment for the patient; or providing behavioral, environmental or contextual modifications for the patient and/or caregiver.

Still referring generally to the system as part of the operating environment 100 of FIG. 1, in an embodiment, at least one or more of the environmental data, behavioral data, physiological data, and contextual data are detected or sensed by an in-situ sensor or in-situ detector 141, 143, 145, 147.

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Results Set No. 1

This study proposes to deploy a low-burden smart health system (BESI-C) to capture the complexity of advanced cancer pain in the home setting from the perspectives of patients and family caregivers. This model could transform how we manage advanced symptoms at home, by being able to monitor, predict, and anticipate distressing symptoms so we can intervene earlier, and more effectively. An aspect of this current study focuses on cancer pain, but the long-term vision is that this model would include many difficult symptoms, such as shortness of breath or nausea, for a variety of advanced stage diagnoses. Specifically, this interdisciplinary research—which combines the expertise of nursing, engineering, medicine, biostatistics, and data science—is innovative in, but not limited thereto, eight key scientific ways: Immediate (this proposal): 1) We know little about patient-caregiver behavioral and contextual variables that influence and predict pain; BESI-C offers a pioneering approach to better understand the complex experience of advanced cancer pain and identify the variables required to develop and deliver timely, tailored, personalized interventions; 2) Research involving patients with advanced disease is challenging, and study procedures must be carefully designed to avoid adding to participant stress. Our proposal offers a solution as sensor technology can collect a wide range of relevant data passively, minimizing invasiveness and burden—a critical consideration for this population, 3) Most smart health interventions rely on ‘apps’ that live on peoples' smartphones. BESI-C is unique in that it is a smart health system that lives in peoples' homes through embedded sensors and a smartwatch and can collect rich, in-depth, data that facilitates personalized system learning, predictive models and effective targeted interventions; 4) The patient-caregiver dyad is understudied in advanced pain symptom research; our study will integrate passively (e.g., heartrate) and actively (e.g., self-reported pain levels) collected data from both the patient and caregiver to better understand how pain may impact the dyadic relationship, and vice versa; 5) participatory research approaches are designed to collaboratively engage key stakeholders to develop solutions that are contextually relevant, and can be especially helpful in designing smart health applications. This proposal is informed by a participatory approach that has incorporated essential patient and caregiver feedback in to the design of BESI-C. Longer-term (future work): Longer term innovations related to BESI-C involve full-scale, real-time data analysis and predictive modeling that can deliver just-in-time notifications with recommended interventions. For example: 6) Patients with advanced cancer who experience uncontrolled pain often seek care in the emergency room out of desperation. BESI-C could reduce unwanted emergency room visits and hospital admissions due to pain; this is especially relevant for patients whose goals of care may include avoiding hospitalization at the end of life; 7) Managing patient symptoms remotely can be challenging; BESI-C can support patients and caregivers who live in rural areas by providing palliative care and hospice providers with real-time data to inform care management decisions; 8) Pharmacological management of serious cancer pain hinges primarily on opioid therapy, which can be highly effective, but also problematic. BESI-C offers a scalable strategy to support patients and caregivers in the safe and effective use of opioids (e.g., by providing guidance regarding dosing and timing of medication) and a platform to monitor for adverse events.

Preliminary Work: This proposal is related to, among other things, system-level approaches to improve cancer pain management. An embodiment of the present inventor's work includes, among other things:
1) BESI-Cancer (BESI-C) Pilot Study #1—Designing BESI-C: In an embodiment, the present inventor envisioned BESI-C for the unique needs of patients with cancer and their family caregivers, with a focus on advanced cancer pain. Specifically, the present inventor created a custom smart watch application that allows patients and family caregivers to mark and characterize pain events from their own perspectives. In an embodiment, the present inventor also included novel environmental and contextual sensing components to the system architecture, such as Bluetooth Estimote beacons to track patient-caregiver location and proximity. The present inventor conducted interviews with cancer patient-family caregiver dyads (n=10) to understand: 1) experiences of cancer pain in the home setting; 2) desired features of the BESI-C system; and 3) variables needed to understand the impact of cancer pain in the home environment. During interviews the present inventor showed dyads BESI-C system prototypes and recorded their feedback about system preferences. The present inventor also presented dyads with a list of potential environmental, behavioral, contextual and physiological variables to measure with BESI-C (based on the literature, clinical expertise of our team, and capabilities of the BESI technology) and asked dyads to rank their relevance to the experience of cancer pain at home. Some important results included, but not limited thereto: 1) dyads view cancer pain at home as a critical issue; 2) dyads are highly receptive to the BESI-C system; 90% of dyads agreed to be re-contacted to participate in a pilot deployment; and 3) dyads confirmed the importance of proposed variables and did not identify additional variables not on inventor's list.
2) BESI-Cancer (BESI-C) Pilot Study #2—Testing Feasibility and Acceptability of BESI-C: In an embodiment, the present inventor has successfully deployed BESI-C with patients with advanced cancer pain and their primary family caregiver dyads (n=5 to date, target 15). With pilot work the present inventor has successfully recruited dyads; streamlined deployment procedures; refined the smart watch interface; verified fidelity of data capture; and generated basic, static templates for sharing data. Through structured interviews and surveys (Table 1), the present inventor have discovered: 1) patients and caregivers find the BESI-C system unobtrusive, helpful and easy to use; 2) dyads will mark and characterize pain events on the smart watch and answer daily survey questions; and 3) participants are eager for opportunities to review and interact with their collected data. Of note, 100% of dyads have completed the full deployment without attrition. Table 1 provides selected results from feasibility and acceptability testing of BESI-C; pilot study #2.
   The pilot work establishes, among other things, proof of concept and feasibility and acceptability of BESI-C. The present inventor has built, tested, and verified the BESI-C system, conducted successful deployments, and are now well-poised to advance BESI-C as described herein.

TABLE 1

| Exemplar Quotes from Semi-Structured Interviews, | | | |
|---|---|---|---|
| Overall Impression of BESI-C (n = 5 dyads) | Selected Likert Survey Results and EMA Metrics (n = 5 dyads) | | |
| It was easy, took little time out of my day . . . I paid more attention | Likert Survey Question | Patient | Caretaker |
| to the small things; it raised awareness about pain management and | (1 = strongly disagree; 5 = strongly agree) | | |
| how she [my partner] looks and acts. (\hat{ }D3C3) | BESI-C can collect helpful information to | 4.6 | 4.6 |
| She [my wife] asked me more specific questions about my pain. (D4P4) | better manage cancer pain | | |
| Made me pay attention to what I was feeling and how my caregiver felt. | Remembering to mark pain events in the | 4.6 | 4.2 |
| (D3P3) | moment was easy | | |
| I think it can help a lot of people out there who cannot get to a doctor | It was easy to answer questions on the | 4.6 | 4.0 |
| when they're really hurting and sick. This is a good way to | Smart Watch | | |
| communicate . . . I think you have a great invention here! (D5C5) | BESI-C made me concerned about privacy | 2.0 | 1.8 |
| Before the BESI-C system I wouldn't always communicate my pain with | BESI-C was a burden for me | 1.4 | 1.6 |
| my caregiver in trying to prevent him from worrying. The system made | EMA Metric | Patient | Caregiver |
| me aware by not communicating I was doing the opposite. (D5P5) | Average time to complete initial pain EMAs | 0:38 | 1:21 |
| \hat{ }D = deployment #; p = patient; c = caregiver | Average time to complete follow-up EMAs | 0:38 | 0:57 |
| | Average time to complete daily EMAs | 1:19 | 2:00 |
| | Percentage of completed follow up EMAs | 63-83% | 44-80% |
| | Percentage of completed daily EMAs | 42-80% | 33-57% |
| | Average # pain events per day/deployment | 3.63 | 1.78 |

### Approach

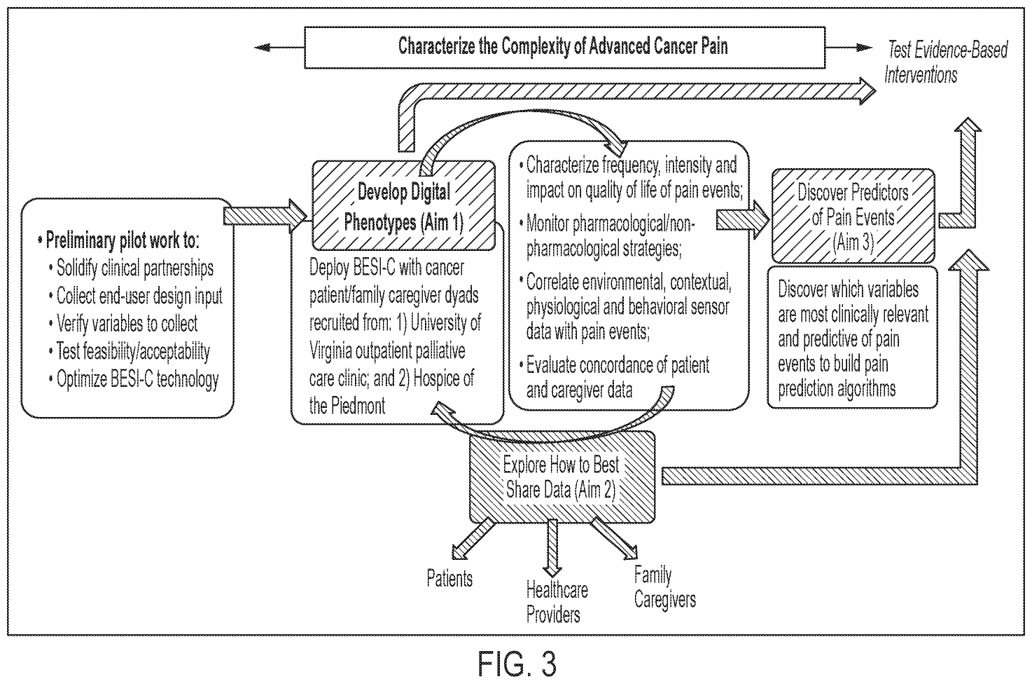

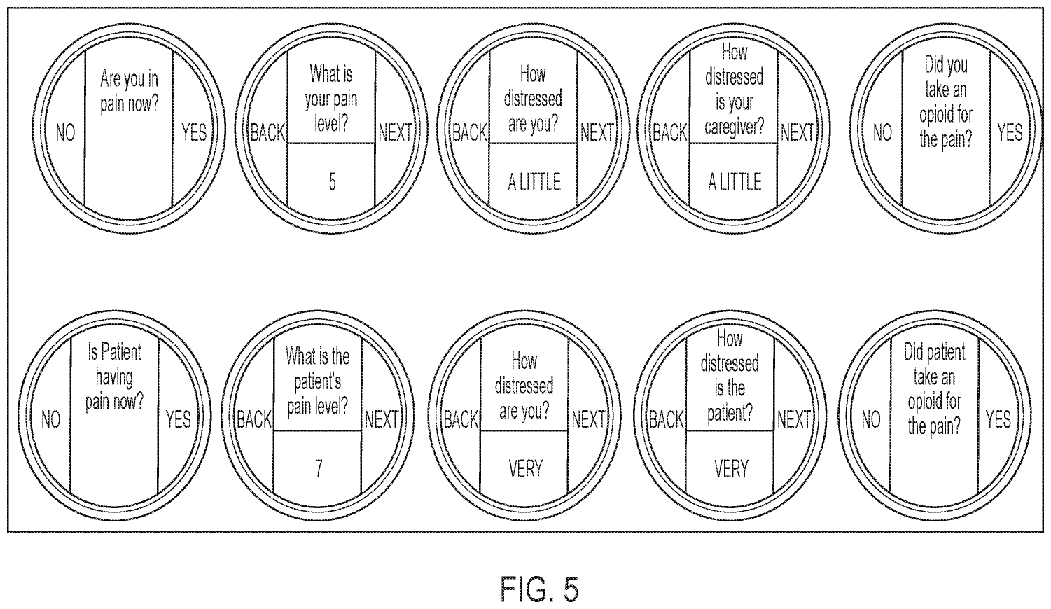

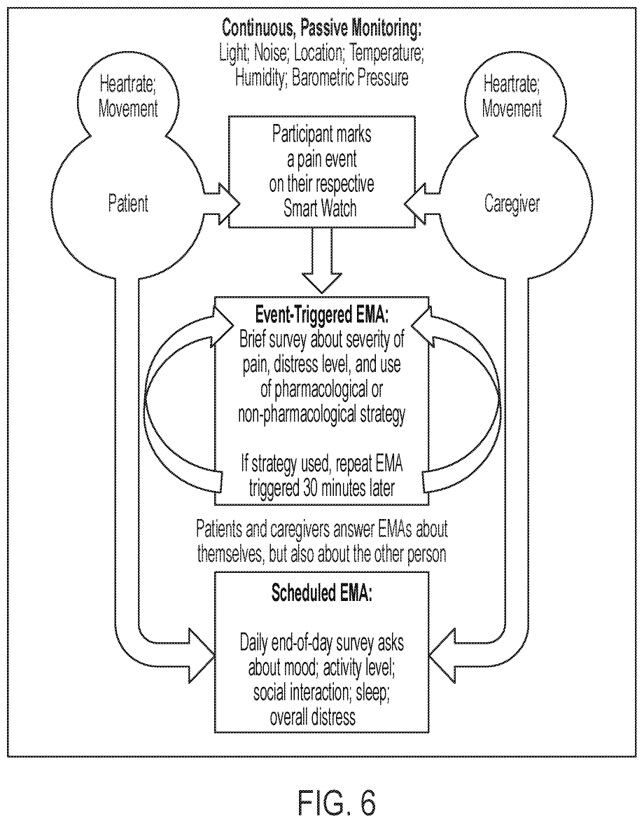

Overall Design: An aspect of an embodiment of the present invention (FIG. 3) deploys a novel package of smart health technology, known as BESI-C, to: 1) describe the complex experience of advanced cancer or non-cancer pain management or cancer-related or other disease-related symptoms in the home setting from the perspectives of both patients and family caregivers; 2) explore optimal ways to share collected data with key stakeholders (patients; family caregivers; healthcare providers); and 3) build predictive pain algorithms to discover which variables are most clinically relevant and predictive of pain events.
Conceptual Frameworks: An aspect of an embodiment of the present invention is grounded in, among other things, three inter-related conceptual frameworks: 1) the Social-Ecological model (SEM); 2) the Dyadic Stress Model and 3) Learning Health Systems. The SEM supports the primary aim of this project, which is to understand the complex interplay of patient, patient-caregiver dyad, and home environment factors that influence the experience of advanced cancer pain. For example, understanding a patient's individual activity and pain levels (intrapersonal level) will involve consideration of dyadic dynamics that exist between the patient and the family caregiver (interpersonal level) that are, in turn, nested within the broader context of the home setting (environmental levels). Levels of the SEM and how they map to relevant variables are summarized in Table 2. Table 2 provides BESI-C variables and sensing modalities. The Dyadic Stress Model posits that life stressors, such as cancer, have a reciprocal impact on patients and their caregivers For example, understanding how patient pain may affect caregiver sleep, and vice versa, is one of the key aspects of this proposal. Learning Health Systems (LHS) have been advocated by the National Academies and the American Society of Clinical Oncology as an effective strategy to achieve timely, sustainable, targeted, and scalable improvement in healthcare delivery. For this proposal, LHS concepts involve the iterative process of collecting data and exploring the most meaningful ways to share the data with stakeholders as a way to learn from behavior and activities.
Rationale for Selected Variables Collected by BESI-C: Variables of interest (Table 2) have been selected based upon, among other things: 1) relevance to pain as identified in the extant literature (e.g., fatigue/sleep); 2) literature documenting the impact of ambient factors, such as light, noise and temperature on the quality of life for palliative care patients; 3) attention to reducing study burden in an already stressed and extremely ill patient population⁰; 4) validation by previously conducted dyad interviews (Preliminary Work); and 5) technology capabilities of the BESI-C system. The BESI-C system will collect active (i.e., requires participant to directly interface with the system by answering a brief question, or mark an event) and passive (collected without user effort) data related to cancer pain from both the patient and caregiver perspective using a cyber-physical platform comprised of wearable devices (smart watches), in-situ sensors and networks, and secure cloud services. BESI-C will collect data at the individual, dyad and home level. For example, at the individual level, patients and caregivers will wear smart watches that passively record information regarding activity and rest; at the dyad level, caregiver-patient proximity/location will be assessed and dyads will be asked to actively mark pain events and report intensity, distress level, and medication or non-pharmacological on their respective smart watch; at the room/home level, environmental sensors will passively monitor factors such as temperature, light and ambient noise. Collecting a range of data, grounded in empirical science, while minimizing participant burden, provides invaluable information to thoroughly understand the complex factors that can influence pain in this vulnerable group—a key scientific gap.
Study Participants and Clinical Sites: The present inventor will recruit participants from two local clinical sites, where present inventor has long-standing relationships: 1) the University of Virginia (UVA) Outpatient Palliative Care Clinic; and 2) Hospice of the Piedmont (HOP). BESI-C deployment and post-deployment assessments will occur within the patient and caregiver's home residence. From each clinical site we will recruit two groups. In an approach, this information may be specific to present inventor's research project; but in an embodiment the invention could be used beyond these settings and patient groups.
Group 1: Patient and Family Caregiver Dyads (Aims 1, 2 and 3). Some key patient inclusion criteria for Group 1 includes adults (age 18 or over) with: 1) a diagnosis of locally advanced or metastatic malignancy; 2) estimated prognosis of at least 1 month (in order to complete study procedures) but less than 1 year, as determined by the patient's primary oncology/palliative care provider using the validated ‘surprise question’ (e.g., “would I be surprised if this patient died within the next year?”); 3) currently taking short-acting prescribed opioids for cancer related pain; 4) an identified primary ‘family’ caregiver; (note: we interpret ‘family’ here in the broadest sense as an informal caregiver who lives full-time with the patient and is involved with their day-to-day care); 5) scores of 6 or higher on NIH PROMIS Cancer Pain Interference scale measures or the Pain Intensity Numeric Rating Scale and 6) cognitive and physical ability to interact with the study smart watch. In an approach, the current research may be focused on adults, but the invention may be used on children.
Recruitment/Enrollment Group 1: In full compliance with Human Subjects procedures, patient and family caregiver dyads will be screened for eligibility and consented either in the UVA Outpatient Palliative Care Clinic or their home residence if enrolled in hospice. If enrolled, baseline demographic and key clinical data (e.g., medication regimen; cancer stage/diagnosis; performance status; pain type/location) will be collected.
Sample Size Group 1: The present inventor will recruit 50 dyads for Group 1 (25 dyads from UVA; 25 dyads from HOP). Sample size is stratified by site as we hypothesize dyads enrolled in hospice and those not enrolled in hospice will be different (see below). Sample size is determined by the minimum number of total pain events per dyad needed for analysis (n=50), and the length of deployment needed to achieve this number. The present inventor proposes 14-day deployments due to: 1) average number of daily pain events based on our pilot data (Table 1); 2) ability to identify potential weekday and weekend differences by capturing two weekly cycles of data for each dyad; 3) our goal to minimize participant burden in this population with a highly dynamic health status; and 4) feedback from dyads during pilot testing. The present inventor considers a ‘pain event’ as when marked by a patient or caregiver, and the one hour time window pre and post the marking of a pain event; any other time period is considered a ‘non-pain’ event. Assuming a conservative coefficient of variation of 0.25 within a dyad, 80% power and alpha of 0.05, we wish to detect any variable that changes by +/−15% during a pain event. This would require 18 dyads per site. This is a conservative sample size since time series data and continuous variables will have more power to differentiate pain and non-pain events than the dichotomous assumption used in this estimate. Allowing for up to 30% of dyads to have incomplete measurements due to technical/compliance issues, or death/physical decline, we will recruit 25 dyads per site, for a total of 50 dyads.
Group 2: Oncology/Palliative Care Healthcare Providers (Aim 2). Inclusion criteria for Group 2 includes healthcare providers age 18 and older involved in the clinical care of patients with advanced cancer pain. Recruitment/Enrollment Group 2: In full compliance with Human Subjects procedures, healthcare providers will be screened for eligibility and consented within the UVA Cancer Center or HOP main offices. If consented, baseline demographic data will be collected. Sample Size Group 2: The present inventor will recruit up to 25 healthcare providers from each site (total of 50 participants); this sample size is based upon the number of palliative care/oncology staff at the two clinical sites and on work exploring data visualizations with participants.
Rationale for Study Sites: The present inventor proposes to recruit from two related clinical sites for a key scientific reason: we hypothesize that the needs and experiences of patients with advanced cancer pain not enrolled in hospice compared to those who are enrolled in hospice will differ. For example, patients are often enrolled in hospice later in their illness trajectory with different pain medication regimens, functional status, and levels of caregiver distress; this may influence how they engage with the smart watch or which variables are most predictive of cancer pain. It is important to note that between 2016-2019 HOP admitted 990 patients with advanced cancer, and the average length of service for these patients was 40 days. It is one of the aims of this study, and in keeping with the objectives of this funding announcement, to better understand the complexity of how advanced cancer pain is experienced in the home context from different illness trajectories. Recruiting from both UVA and HOP will allow us to examine similarities, and differences, between the dyad experience of advanced cancer pain experienced by non-hospice and hospice dyads.
Data Collection and Analysis Procedures: To understand data collection and analysis procedures, it is useful to detail the architecture of the BESI-C system, which includes: (FIGS. 4 and 5):
1) SMART WATCHES (Wear OS Fossil Sport Watch): worn by both the patient and the family caregiver to collect both passive sensor data (photoplethysmogram heart rate and motion data via accelerometer and pedometer) and active Ecological Momentary Assessment (EMA) data. In an embodiment, the present inventor elected to use a commercial off-the-shelf smart watch, as we prioritized wearability of the device with the acknowledgement we are not currently using collected data to direct or alter clinical care. EMAs are brief, contextual assessments commonly used in mobile health to measure symptoms in real-time. Each smartwatch is programmed with a custom BESI-C application (‘app’) designed for either the caregiver or the patient. The BESI-C custom smartwatch app includes both event-triggered and scheduled EMAs. Event-triggered EMAs allow patients and caregivers to independently mark pain events and record pain severity, perceived distress, opioid medication use, and use of non-pharmacological strategies. Scheduled EMAs are automatically generated once daily and ask a brief series of “1-click” questions regarding mood, sleep quality, activity level, and amount of social interaction. Iterative design of the BESI-C custom app has prioritized ease of user interface, speed and simplicity in completion of EMAs, and low burden and interference with activities, such as sleep. Dyads are asked to wear the watches as much as possible (preferably 24/7) during deployment and are given 2 watches to swap out when battery life decreases.
2) SENSOR RELAY STATIONS: custom-built environmental sensor stations are strategically deployed in each primary room of the dyad home to passively and continuously collect data on room-level temperature, light, humidity, barometric pressure and ambient noise. In an embodiment, a standard sensor placement protocol is used that considers sensor range and size/configuration of the room. “Primary” rooms include those where participants tend to spend the most time and generally are the living room, bedrooms and kitchen. Environmental data streams are integrated and transmitted to the base station (FIG. 4).
3) BLUETOOTH BEACONS: commercially available Bluetooth Low Energy Estimote Beacons that continuously broadcast device identification information are deployed strategically in the dyad's home, and their broadcast signals are received by the smartwatches. Using the smartwatches' received signal strength indicator (RSSI), the BESI-C app can determine the wearer's distance from each beacon, thereby enabling room-level localization of the wearer and an estimation of patient-caregiver proximity.
4) BASE STATION: a BESI-C configured laptop is placed in an unobtrusive location within the dyad's home to provide a cyber-physical platform for data offloading and remote system monitoring.
   Privacy and data security have been carefully considered and are addressed in the following ways: 1) the BESI-C system does not record raw audio data, only pre-processed features related to ambient noise characteristics that do not enable reconstruction of conversation content; 2) the system contains no cameras; 3) sensors are only deployed in rooms approved by the participants and never in highly personal areas, such as bathrooms; 4) participants can turn off sensors at any time, simply stop wearing the smart watch, or put the smart watch in to a temporary ‘do not disturb’ mode; 5) all data streams are de-identified, contain no patient identifiers and are labelled only with a study identification number; 6) all data are streamed to a base station laptop via a local Wi-Fi network with a dedicated router and stored on a secure S3 bucket on a commercial cloud service (AWS) via a secure API access key. Of note, Internet access allows remote system monitoring, but is not required for actual data collection. If patients or caregivers are outside of the home, they can still enter data on their smart watch, which is stored locally on the watch until the participant returns home and is re-connected to the BESI-C network. If a dyad does not have reliable internet access in the home, a mobile hot-spot is set up to allow remote system monitoring. The environmental sensors and localization beacons are installed in the patient's home and are not re-located (for example if patient is admitted to the hospital), as we are interested in capturing the home context and how that may influence pain. However, the wearable sensor (smart watch) will continue to collect data regardless of participant location.
   AIM 1: Develop comprehensive digital phenotypes of advanced cancer pain in the home setting. The present inventor conceptualizes ‘digital phenotype’ as introduced by Torous et al. as the “moment-by-moment quantification of the individual-level human phenotype in-situ using data from smartphones and other personal digital devices”, and expand this definition by considering the family caregiver and dyad level as well. In Aim 1 the present inventor strives to answer the research questions: What does the experience of advanced cancer pain in the home setting look like from the perspective of individual patients and family caregivers, and also as a dyad? What behavioral, physiological, environmental and contextual factors (see Table 2) may precipitate, influence and modulate cancer-related pain events (or for example non-cancer related pain events)? The present inventor hypothesizes that individuals, and dyads, will display a unique ‘digital fingerprint’ of the cancer pain experience—that if better understood can be utilized to inform and deliver personalized, evidence-based interventions. The present inventor also hypothesizes that BESI-C will be particularly effective in helping characterize and better understand the phenomenon of breakthrough cancer pain—pain that increases or ‘spikes’ above baseline pain—and that is notoriously difficult to predict and manage.
   Data Collection: With prior pilot work (see Preliminary Work section), in an embodiment the present inventor have established and streamlined deployment procedures. Day 0: Set-up of the BESI-C system is completed by a team of trained engineers and nurses, takes approximately 1.5 hours and involves: 1) patient and caregiver education; 2) placement of relay sensor stations and Bluetooth beacons; and 3) base-station set-up and verification of data streams. BESI-C will be deployed in the homes of participant dyads for a maximum of 14 days (see above, Sample Size, Group 1). Passively collected physiological (heartrate, step count), environmental (light; temperature; barometric pressure; ambient noise) and localization data are continuously collected without any interaction needed by the patient or caregiver. Actively collected behavioral and contextual data involve the caregiver or patient interacting with their smart watch to mark the time of a pain episode and describe the pain event (FIG. 6). For example, patients or caregivers are asked to either push a button or tap the screen on the smart watch when the patient is experiencing an episode of cancer pain, as perceived by the respective participant. We recognize that identifying discrete cancer-related pain events may not always be straightforward; we therefore educate participants to consider a ‘pain event’ as ‘one in which the pain has increased from what it was previously and that you feel requires attention.’ If a pain event is clearly unrelated to the patient's cancer (e.g. stubbing a toe) we explain that these events do not need to be recorded. If a pain event is marked, this generates a brief EMA which asks the participant to rate the severity of pain on a simple 0 (no pain) to 10 (worst pain imaginable) scale, their distress level, their perceived partner's distress level, and if any opioid pain medications were taken or non-pharmacological measures employed. If using a pain alleviating strategy is reported, a repeat EMA is automatically deployed to the participant's smart watch approximately 30 minutes later to see if pain has decreased. If a participant indicates an opioid was not taken for a pain event, we ask them to tell us why (e.g., not time yet; concerned taking too much medication; side effects; pain not bad enough; out of pills; some other reason). Additionally, a brief end-of-day scheduled EMA survey (approximately 10 questions) asks participants to rate their activity, mood, sleep, social interactions and overall pain and distress levels over the past day and is used to corroborate passively collected data streams. For example, if a patient reports being ‘very active’ in their daily EMA survey, we can corroborate this with passively collected accelerometer, localization, and step count data. It is important to note that we have intentionally elected to use simple, rapid EMA assessments (versus, for example, digital adaptations of longer, validated pain assessments) because: 1) we are asking participants to pause and mark pain events in the moment and our current feasibility study indicates this must be extremely easy and quick to do; 2) we need an EMA strategy that works on a smart watch in terms of a simple, readable user interface; and 3) the validity of using single item measures for symptom assessment has been previously established. Dyads are also asked to keep a simple, daily log to record any significant clinical or unusual events that may occur during the deployment, such as a fall/injury. Importantly, because BESI-C will not currently alter or direct patient care or medication use participants are carefully counseled to follow standard procedures for notifying their care team if they experience concerns or changes with their health status. Days 1-14: The deployment is remotely monitored, and dyads receive periodic phone check-ins from the study team. In-coming data is used to generate data visualizations. Day 15: The study team will remove the BESI-C equipment and conduct a brief structured interview and survey with dyads using the well-validated System Usability Scale (SUS), which contains 10 standard and validated questions assessing systems usability. Data visualizations will be shared and evaluated (see below, Aim 2).
   Data Analysis: An embodiment will use principles of signal processing and machine learning to develop comprehensive digital phenotypes of advanced cancer pain in the home setting from three unique viewpoints—patients with advanced cancer; family caregivers; and patient-family caregiver dyads—nested within two groups recruited from: 1) hospice and 2) an outpatient palliative care clinic (i.e., not enrolled in hospice). Specifically, we will: a) characterize the frequency, intensity and impact on quality of life (mood; sleep; social interaction; activity; overall distress) of pain events; b) monitor the use of pharmacological and non-pharmacological strategies and self-reported efficacy; c) correlate or relate environmental, contextual, behavioral and physiological data with reported pain events; and d) evaluate concordance of patient and caregiver data. For example, we can explore if pain events are equally marked by caregivers and patients, how they are respectively characterized in terms of severity and perceived burden, and how this corresponds with medication use, non-pharmacological strategies, mobility, sleep, heartrate and home/room level (e.g., temperature, light, noise) data. One of the goals of Aim 1 data analysis will be to explore the ability of the sensing modalities to identify patterns, relationships, and concordance between actively and passively collected data. Concordance will be measured for each dyad as an intra-class correlation (ICC). Concordance will be dichotomized (yes/no) within 30 minute epochs of time, and logistic regression used to determine if concordance is affected by any measured characteristic (such as severity and perceived burden, medication use, non-pharmacological strategies, mobility, sleep, heartrate and home/room level data) or if concordance improves over time within a dyad, and if mixed effects models detect similar concordance patterns across dyads. C-statistics will be used to measure the dyad calibration across continuous measurements. In an embodiment, we are planning on focusing analysis on severity of pain events (those marked as 5 and with corresponding moderate/high levels of distress) and frequency of pain events (number of marked pain events in a specified time period, regardless of severity/distress level). The number of pain events will be compared between patients using mixed effects Poisson regression (which can account for potentially variable amount of data collection time and look for similarities across dyads), and to test for differences in rates by demographic, clinical and environmental characteristics. Sensor data will be summarized over time using standard measures, such as mean, variance, max, min, for inclusion in the regression models. Structure equation models may be used to explore associations and develop hypotheses between variables that have recursive relationships. Table 3 lists a selection of example analysis questions and hypotheses (not an exhaustive list) we propose to use to create the digital phenotypes. Table 3 provides example data analysis questions and hypotheses to develop the digital phenotypes of advanced cancer pain. Referring to Table 3, in an embodiment, the term/activity pertaining to “relate” or “relating” may be implemented in instances wherein “correlate” or “correlation” is listed throughout Table 3, respectively.

TABLE 3

| Question | Variables | Hypothesis/Comments |
|---|---|---|
| What environmental factors | IV¹: Room temperature; light; noise, humidity, | ↑ temperature, light, noise. barometric pressure and |
| correlate with frequency of patient | arometric pressure; room location; patient/caregiver | humidity will ↑ frequency and severity of pain events; |
| and caregiver marked pain events, | proximity | certain room locations and ↑ pattern and caregiver |
| particularly those rated as ≥5 | DV²: Number of marked pain events with severity | proximity will ↓ frequency or severity of pain events |
| severity? | of ≥5 in a 24-hour period | |
| What behavioral/physiological | IV: Activity/step count: sleep quantity and quality; HR; | ↓ sleep quantity and quality will ↑ frequency and |
| factors correlate with frequency | use of pharmacological/non-pharmacological strategy | severity of pain events; ↑ activity/step count = ↑ |
| of patient-marked pain events, | DV: Number of marked pain events; pain events rated ≥5 | frequency and severity of pain events |
| particularly these rated as >5 | | |
| severity? | | |
| What behavioral/physiological | IV: Activity/step count; sleep quantity and quality; HR; | ↓ sleep quantity and quality will ↑ frequency and |
| factors correlate with frequency of | use of pharmacological/non-pharmacological strategy | severity of pain events; ↑ activity/step count will ↓ |
| caregiver-marked pain events | DV: Number of marked pain events; pain events rated ≥5 | frequency and severity of pain events |
| particularly those rated as ≥5 | | |
| severity? | | |
| How does physical proximity | IV: Beacon localization of patient/caregiver interaction | ↑ caregiver/patient proximity = ↑ proportion of |
| influence concordance between | DV: Number of matched patient and caregiver marked | concordance of marked pain events |
| patient, marked pain events and | pain events³ | |
| caregiver marked pain events? | | |
| When there is a matched marked | IV: Matched marked pain event | Particularly interested in how patients perceive/rate |
| pain event, what is the concor- | DV: Patient and caregiver ratings of pain severity; | caregiver distress and how caregivers perceive/rate |
| dance between patient and | distress; whether they took medication and if they | patient distress |
| caregiver repeat EMA responses? | needed additional medication | |
| What is the correlation between | IV: Mood, activity level; social interactions; sleep | Patients and caregivers will mark ↑ pain events more |
| marked pain events and daily | quality/quantity; overall pain; overall distress | frequency and of higher severity with ↑ medication |
| EMA survey responses for both | DV: Frequency of marked pain events, severity of pain | use on days they report ↓ mood ↓ sleep quality/ |
| patients and caregivers? | events, frequency of medication use, distress levels | quantity, |
| | | ↓ social interaction, ↑ overall distress levels and ↑ |
| | | overall pain interface levels |

¹IV = independent variable;
²DV = dependent variable;
³Matched pain events are those marked independently by patient and caregiver within approximately 30 minutes at each ether.

AIM 2: Explore and evaluate preferences for communicating collected data with patients, family caregivers and healthcare providers. Visually representing complex and diverse patient (and caregiver) generated data in an understandable and meaningful way can help inform care decisions and improve care outcomes. However, how to best create data visualizations—particularly for large amounts of health related, heterogeneous self-monitoring data—is unclear, and a critical research need. Significant work related to data visual analytics has been done in chronic care disease management, such as diabetes; to our knowledge this would be the first exploration of data visualizations specifically related to advanced cancer pain from the dyadic perspective of patients and family caregivers. In Aim 2, we strive to answer the research questions: What is the most meaningful way to visually represent ‘digital phenotype’ data related to advanced cancer pain and convey that to key stakeholders (patients, family caregivers, healthcare providers)? The present inventor hypothesizes that different ‘buckets’ of data exist, and who needs access to these data—when, and how, and in what ways—will vary, temporally and by end-user. For example, there are likely data most relevant for the patient themselves; data best mutually shared between patients and family caregivers; data helpful for the caregiver only; data best shared between healthcare providers and family caregivers; and data most helpful to healthcare providers. Aim 2 seeks to better understand these complex data sharing preferences.
Creating the Data Visualizations: Extracted features from data collected during BESI-C deployments will be visually represented using software such as R using the shiny package to create an interactive web-based visualization. R is a programming language and free software environment for statistical computing and graphics supported by the R Foundation for Statistical Computing. The interactivity of the application will focus on allowing the user to alter the granularity, data elements being visualized, style of presentation, and time period being shown. These applications will also contain a package that can track the user's interactions with the application—for example, what elements were most interacted with, and the order of use—, to allow a quantitative assessment to help further optimize the visualization. Deciding which extracted features to highlight and present is one of the key objectives of this aim, and we will use a user-centered participatory approach to understand which visualization elements are most helpful to present data regarding advanced symptoms to patients, family caregivers, and healthcare providers.
Data Collection: In an embodiment, the present inventor will share data visualizations via an iPad with patient/caregiver dyads and healthcare providers. Feedback will be gathered by: 1) allowing users to interact with the visualizations and recording unstructured ‘think aloud’ feedback; 2) a structured interview; and 3) a brief Likert-style survey that will ask participants to agree or disagree with statements such as, ‘I found the data summary easy to understand.’ Interview questions will address: 1) perceived utility/relevance and comprehension; 2) data content and display preferences; and 3) data sharing/access preferences (Table 4). Table 4 provides example questions to evaluate data visualizations with stakeholders. For patient/caregiver dyads (Group 1) we will collect feedback regarding data visualizations when BESI-C is removed from the dyad home (Day 15). Healthcare provider participants (Group 2) will be asked to participate in a 1-hour session to provide perspectives on data visualizations. We are planning that these sessions may be one-on-one sessions, or a larger group format, depending on timing, logistics, and preferences of busy clinicians. For healthcare providers, we will host 3 sessions at different time points over the course of the study to gather iterative feedback. Between each time point the inventor may work to iterate and refine the data visualizations.

TABLE 4
Questions for Patients/Caregivers/Healthcare Providers

Perceived Utility/Relevance
Does this summary capture the information you are most interested in?
What information do you find most helpful and why? Least helpful, and
why?
Is there information you would like to see that is not included in this
summary?
Will this information change how you manage your pain? If so, in what
ways?
Would this information help you guide a patient's plan of care? If so,
in what ways?
Does this summary accurately represent your experience? The
experience of your partner?
Data Content and Display Preferences
What information, if any, would you like shared with your healthcare
provider?
Who else would you like to see this information and why?
Is the data pleasing to look at? (e.g., shapes/colors/fonts)
Date Sharing and Access Preferences
How would you like this information to be shared with you? (e.g., paper
print out; website; mobile app)
Do you think you need to see different information than your partner?
If so, can you provide some examples?
When, ideally, would you like to see this information?

Data Analysis: ‘Talk aloud’ comments and structured interview responses will be collated and analyzed using an inductive thematic approach to identify patterns expressed by participants. Responses will be compared across (e.g., caregiver to patient) and within (e.g., caregivers only) participant groups. Likert survey results will be analyzed using descriptive summary statistics. Usage patterns of the interactive application will be simplified to time course of different events based on the elements of the visualization displayed, and analyzed for the duration and order of these events. Usage patterns for each dyad and healthcare provider feedback session will be compared with Likert survey, ‘talk aloud’ feedback, and interview results to identify both useful and problematic elements in the visualization, as well as identifying the optimal ordering and visual display of various data elements. Improvements will be incorporated in to subsequent revisions of the data visualizations.
AIM 3: Discover which sensing data are most predictive of pain events to build parsimonious pain prediction algorithms. In Aim 3, we strive to answer the questions: can we predict when pain and distress are most likely to occur with patients and family caregivers? Which variables and sensor data are most clinically relevant and predictive to identify the precursors and moderators of breakthrough pain events? What do pain prediction algorithms look like for individuals and specific dyads? Ultimately, we aim to understand and identify which data variables are most predictive in identifying breakthrough pain events so we can: 1) streamline the BESI-C data collection system; (e.g., we may discover, for example, that light is not highly predictive and we can omit that from our sensing platform); and 2) inform evidence-based pain alleviating interventions for both patients and caregivers to test in future clinical trials.
Data Collection: All passive streaming data from environmental and smartwatch sensors will be integrated with active EMA data collected from patients and family caregivers (see Data Collection, Aim 1).
Data Analysis: One of the primary outcomes for Aim 3 data analysis will be pain events. An event will be classified as a pain event when it is marked by either the patient or caregiver. One hour pre- and one hour post-pain event marking will be classified as part of that pain event. Non-pain events will include any time not marked as a pain episode. Using principles of functional data analysis, advanced machine learning and multiple time series analysis we will take the first step towards building parsimonious pain prediction algorithms. We will follow the procedures below in a hierarchical fashion using patient data; caregiver data; and patient plus caregiver dyadic data. First, we will analyze the dichotomous outcome of pain events using 30 minute epochs of time for each dyad separately. Each measured characteristic will be evaluated independently for its predictive ability using a range of techniques as appropriate for the measurement under consideration. This will include machine learning methods for time series, frequency and spectral analysis, and regression analysis. Time series measurements may have a lagged prediction, which will be tested for. Measures showing predictive promise for an individual dyad will then be combined into a multivariable predictive algorithm for each patient to describe the most predictive measurements and look for similarities across patients. Next, the multiple epochs from each dyad will be combined to assess the association between measured characteristics and pain. These analyses will be stratified by hospice and non-hospice patients. Should similarities be observed between strata, we will explore if they can be combined. Associations will be assessed using longitudinal data analysis techniques, and/or machine learning techniques for time series. Finally, the entire data stream for each dyad will be analyzed in a multiple time series analysis via functional data analysis techniques. Functional data analysis will be able to use continuous time and identify periods where measures are predictive of an event, allowing us to revise the epoch of time used as needed for each measure investigated, as well as visualize the pattern of each measure over time. Complex interactions between measurements and pain will also be explored through interaction terms, cluster analysis, and factor analysis for all methods investigated. The best predictors will be combined in a multivariable model and the sensitivity, specificity, positive and negative predictive values calculated.

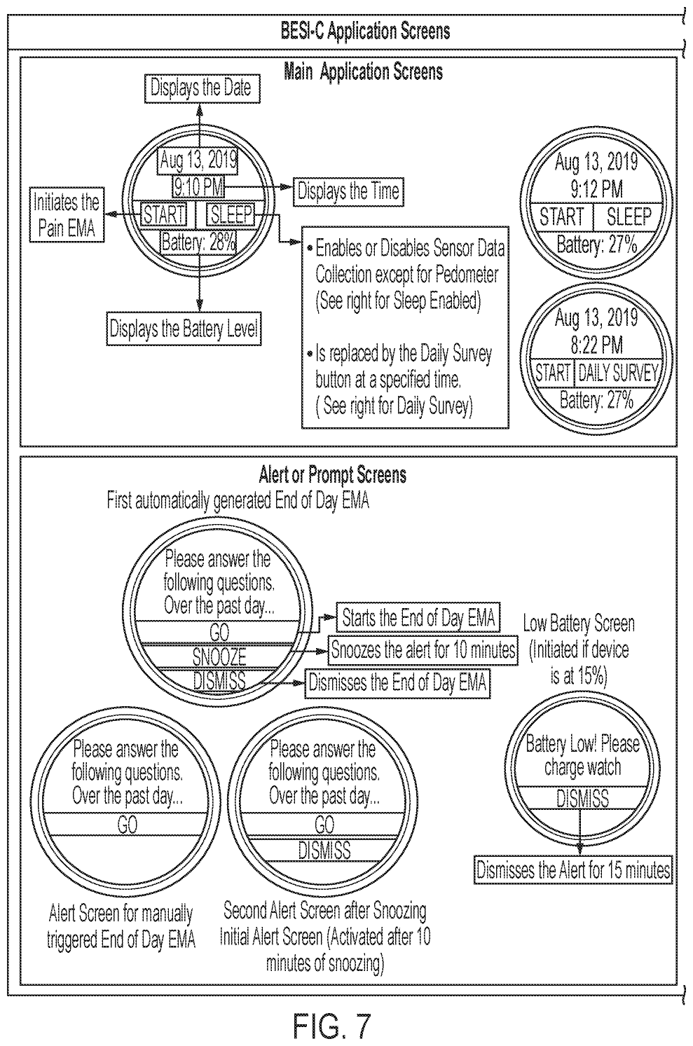

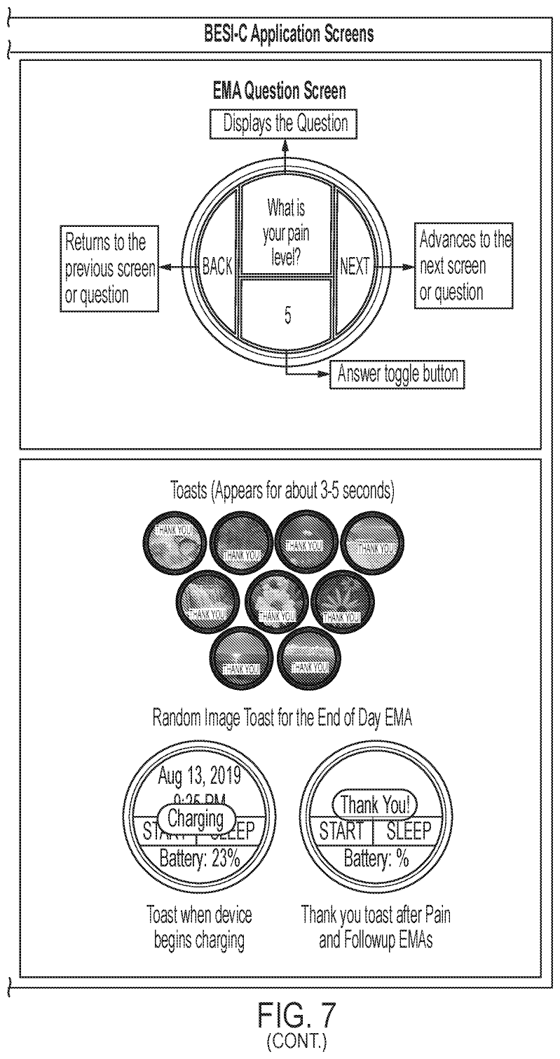

Turning to, FIG. 7, FIG. 7 provides a screenshot of selected examples of “BESI-C Application” screen displays, in accordance with certain example embodiments.

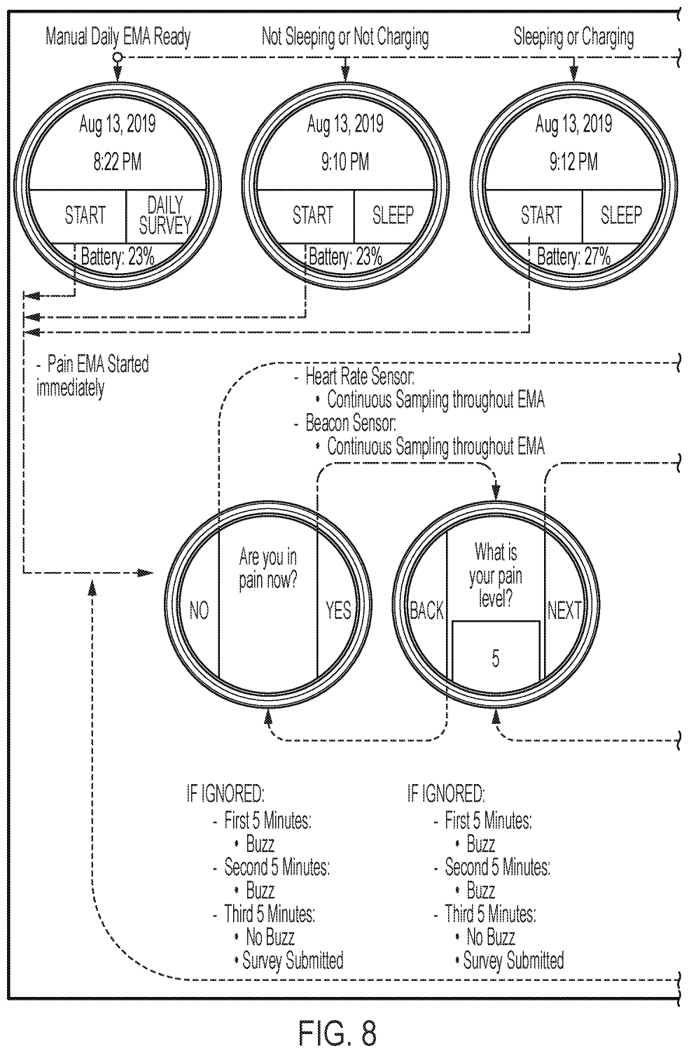

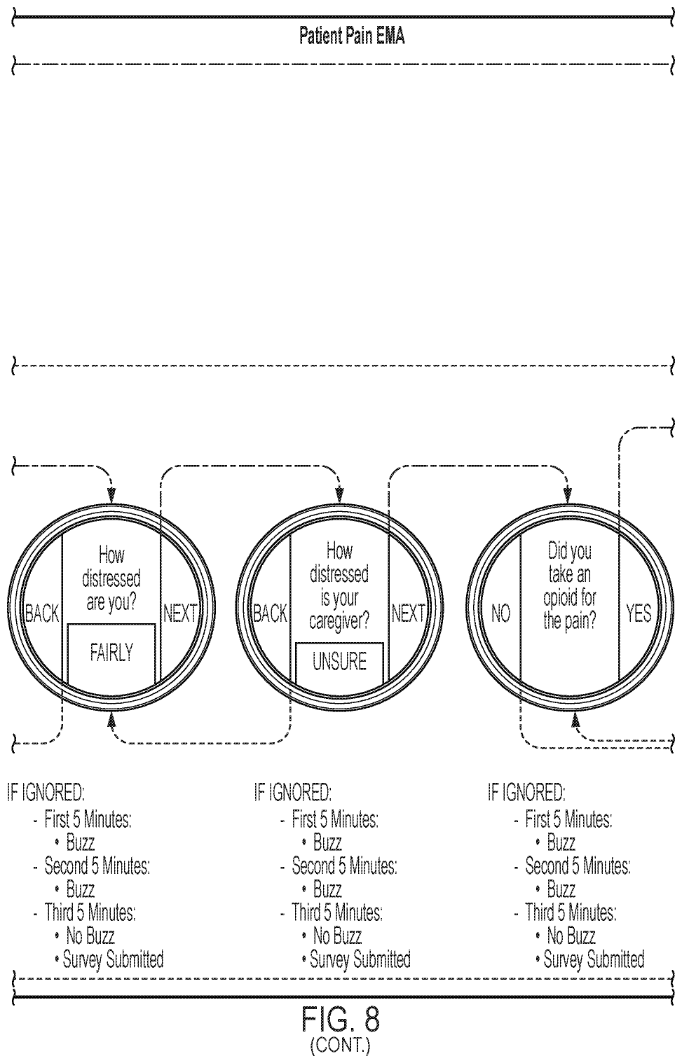

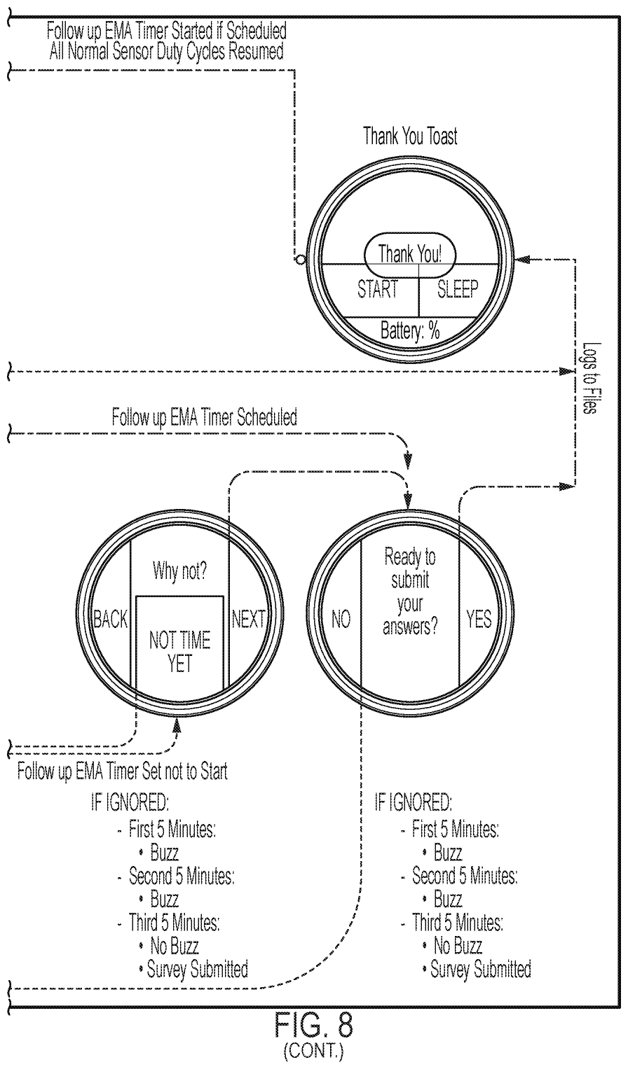

Turning to, FIG. 8, FIG. 8 provides a screenshot of selected examples of “Patient Pain EMA” pertaining to, for example but not limited thereto, how a patient initially marks and describes a pain event, in accordance with certain example embodiments.

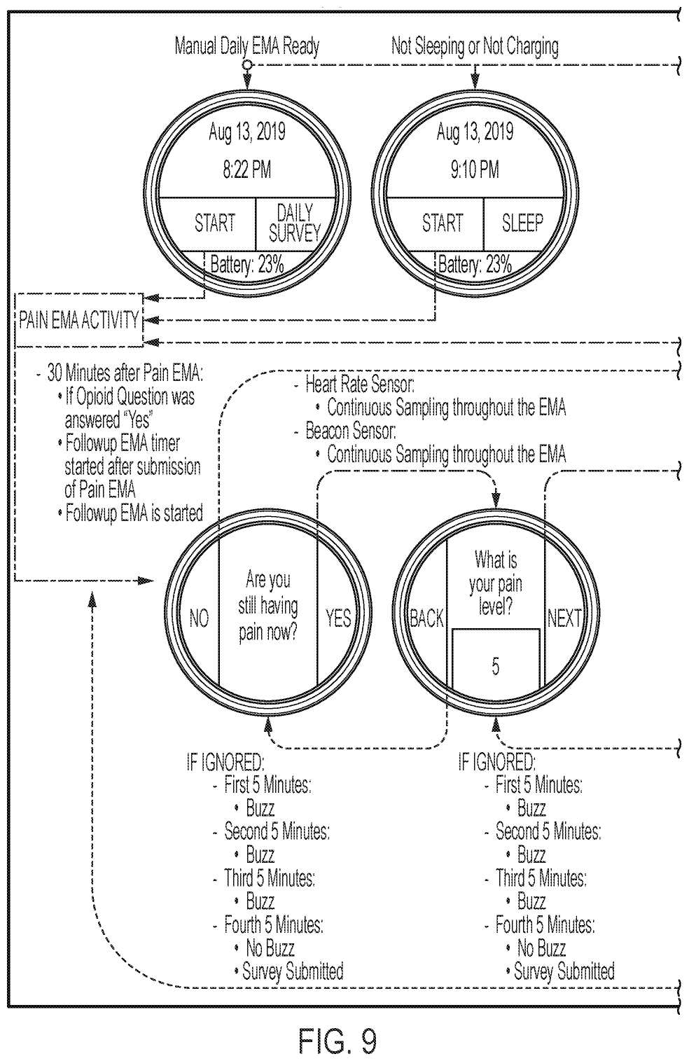

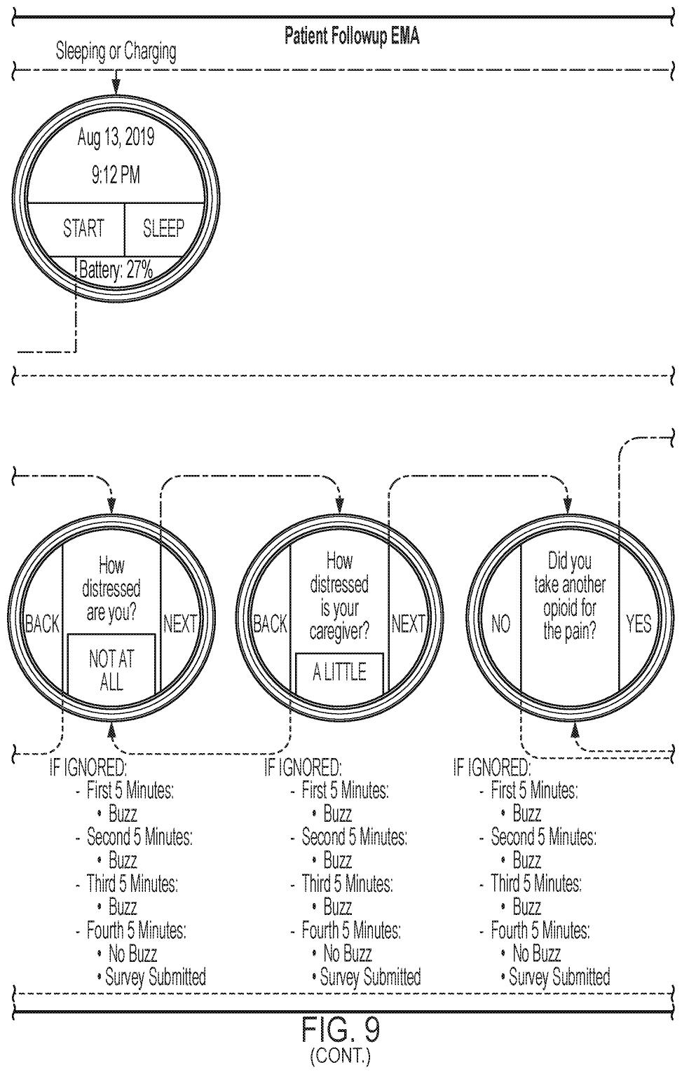

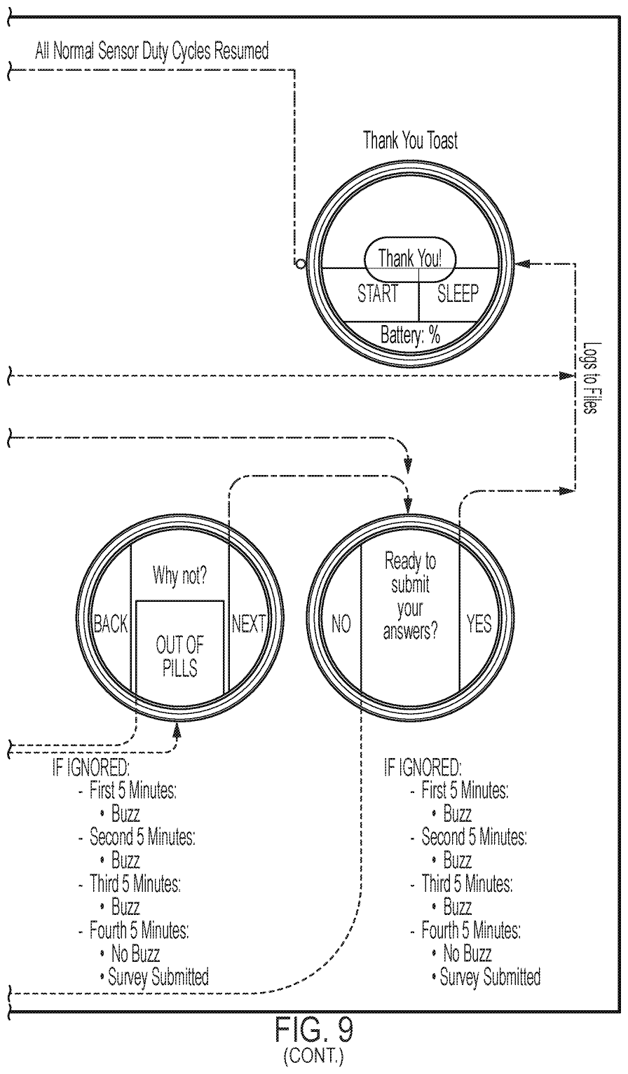

Turning to, FIG. 9, FIG. 9 provides a screenshot of selected examples of “Patient Follow-up EMA” pertaining to, for example but not limited thereto, how a patient describes pain 30 minutes after using a pharmacological or non-pharmacological strategy to reduce pain, in accordance with certain example embodiments.

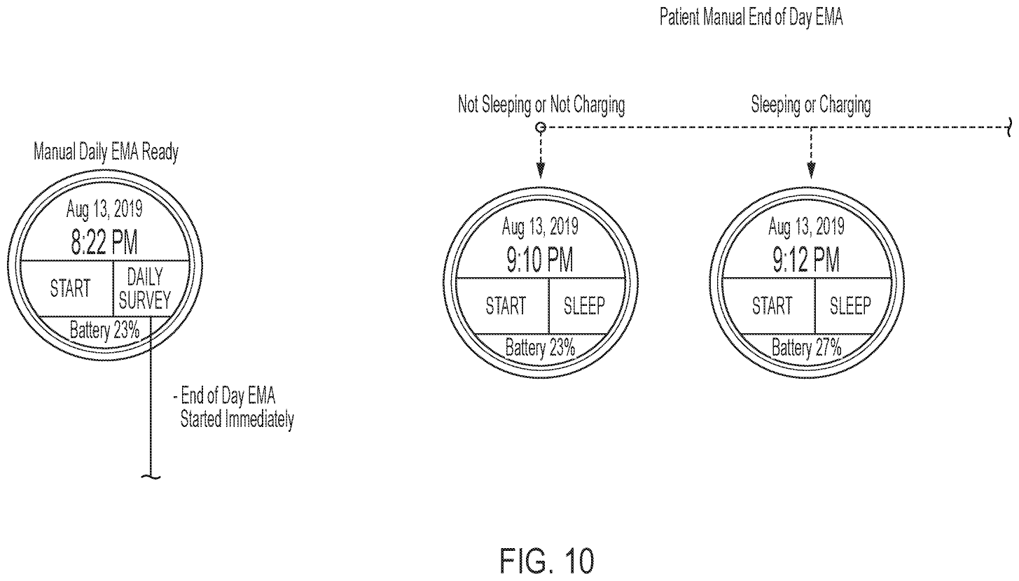

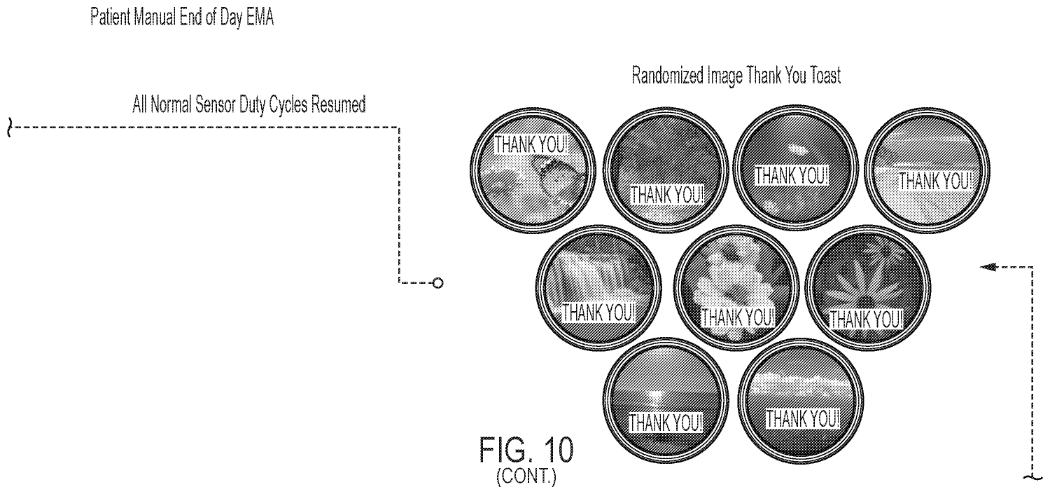

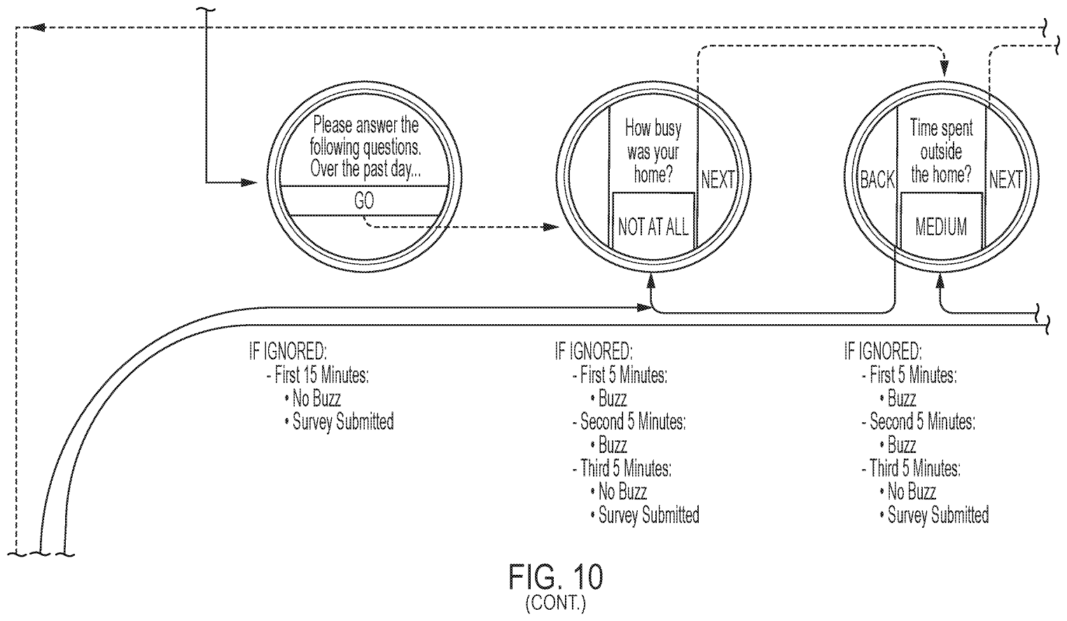

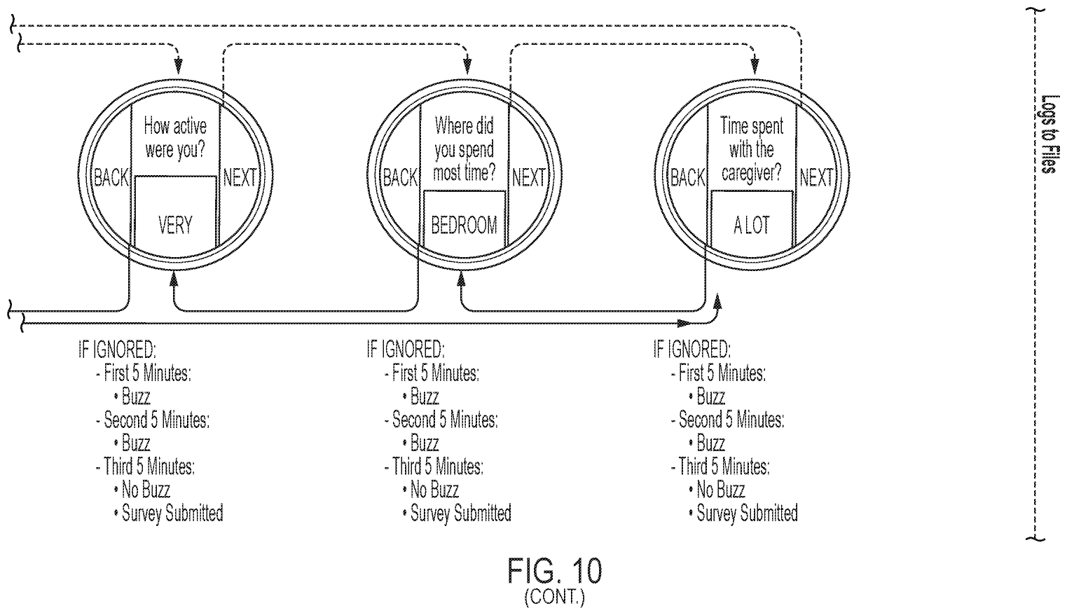

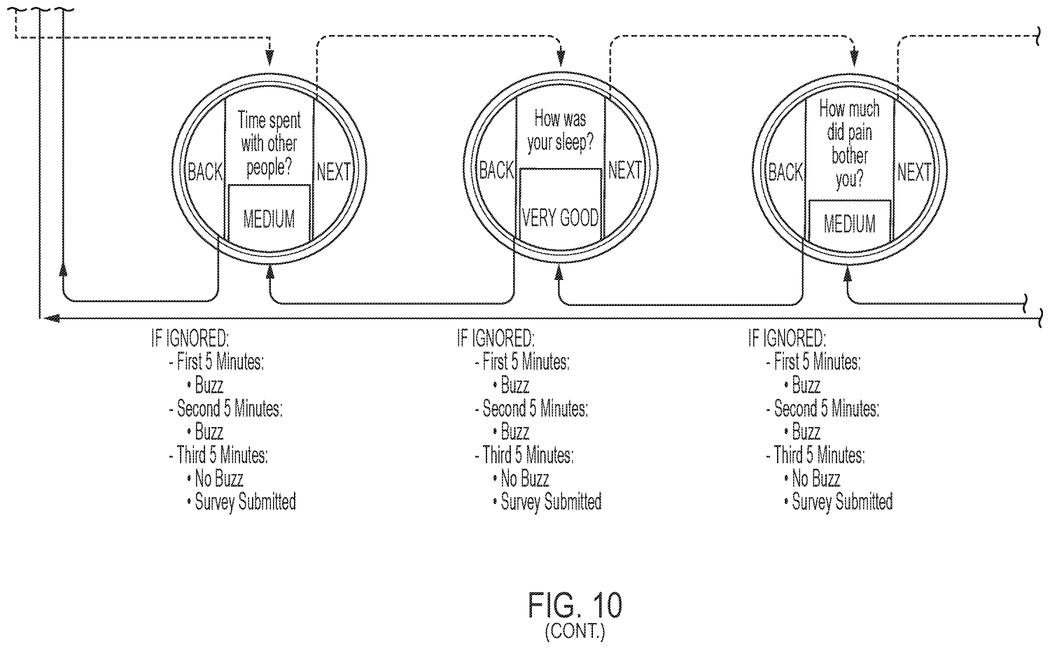

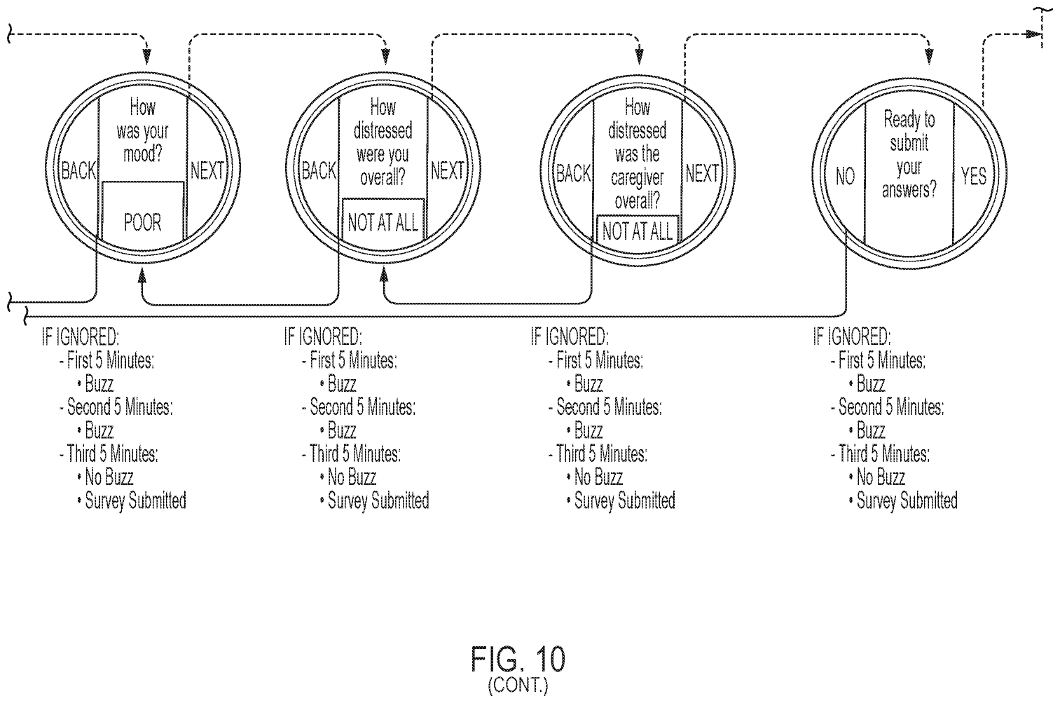

Turning to, FIG. 10, FIG. 10 provides a screenshot of selected examples of “Patient Manual End of Day EMA” pertaining to, for example but not limited thereto, a patient survey at the end of the day that assesses general patient well-being, contextual factors and behaviors, in accordance with certain example embodiments. In an embodiment, the patient manually generates this survey at time of their choice after 5 pm.

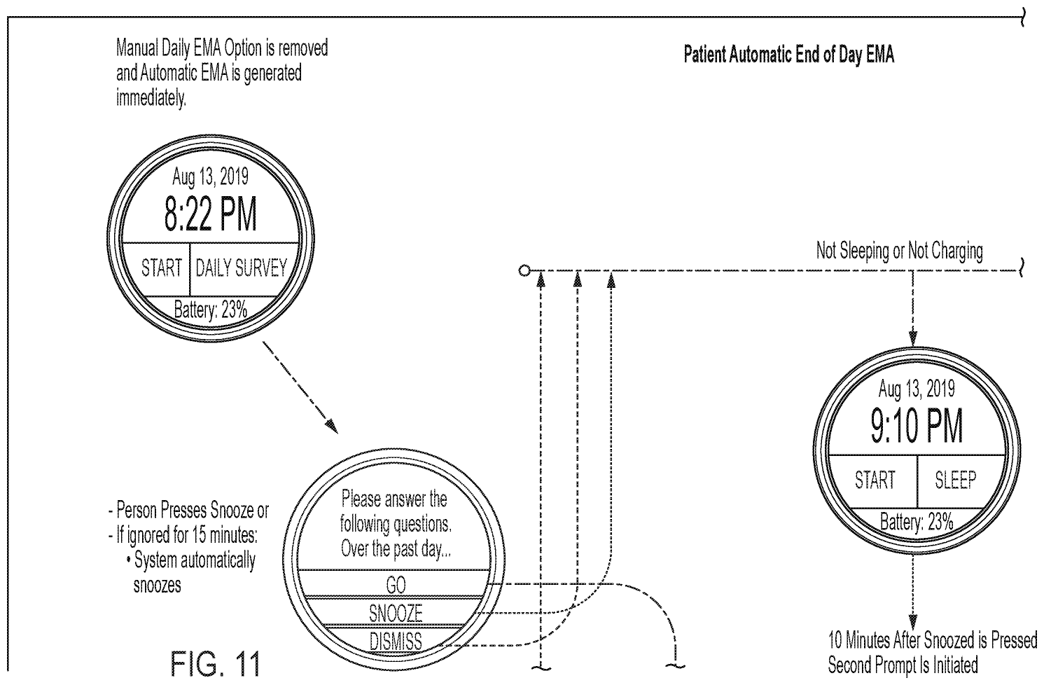

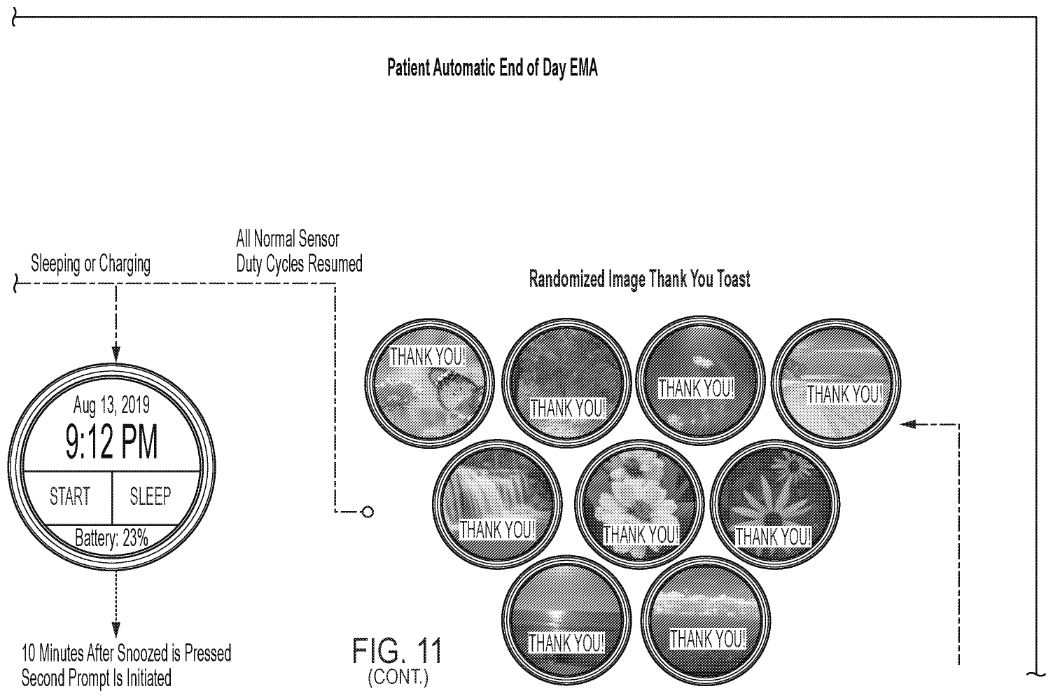

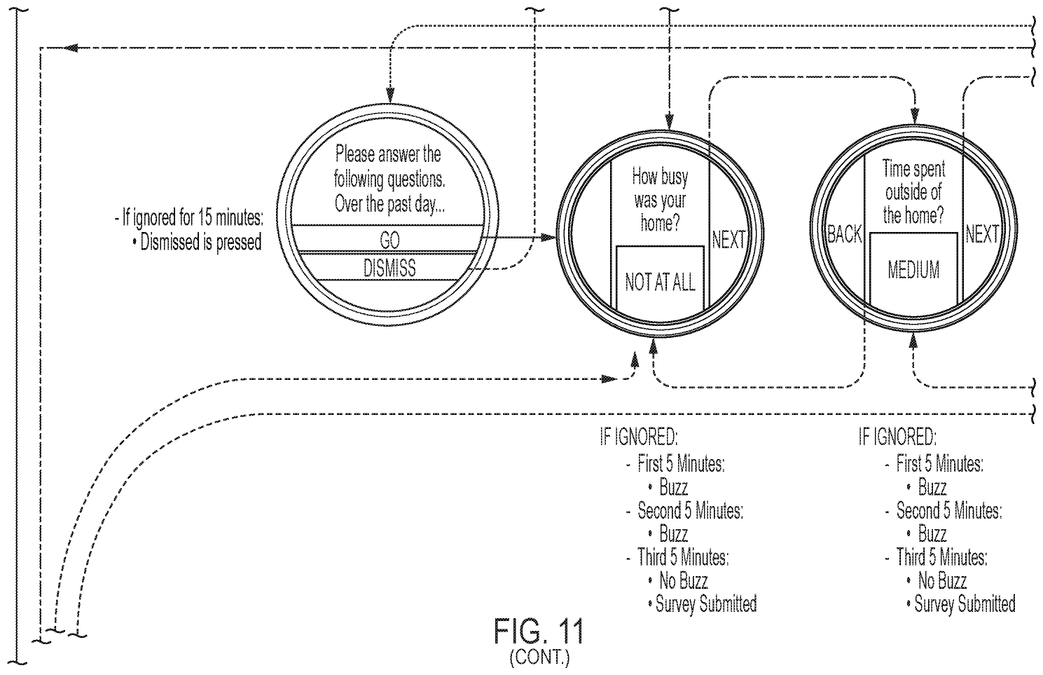

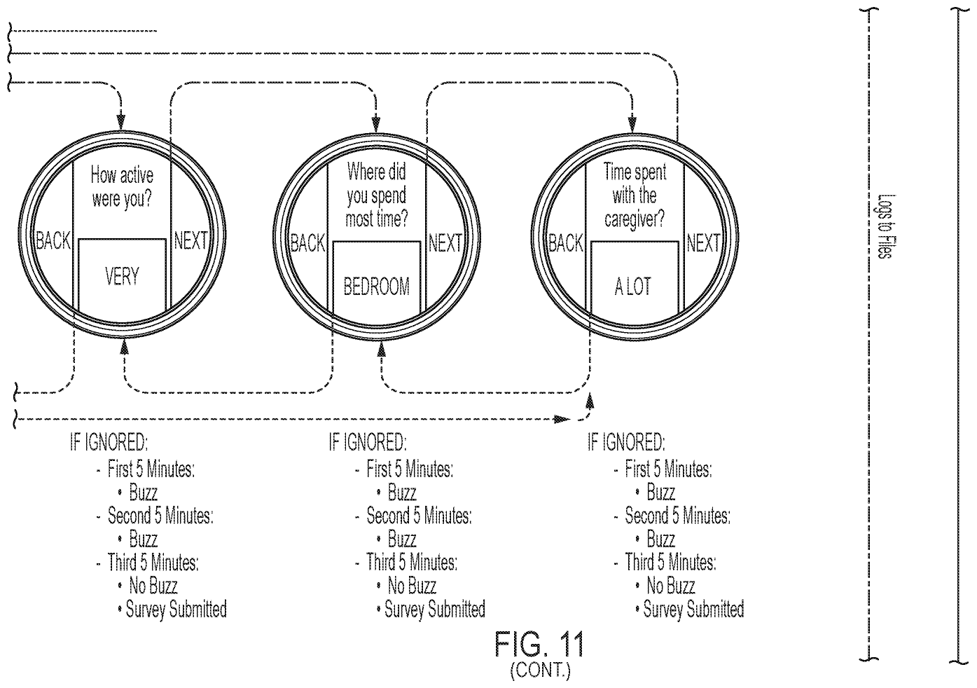

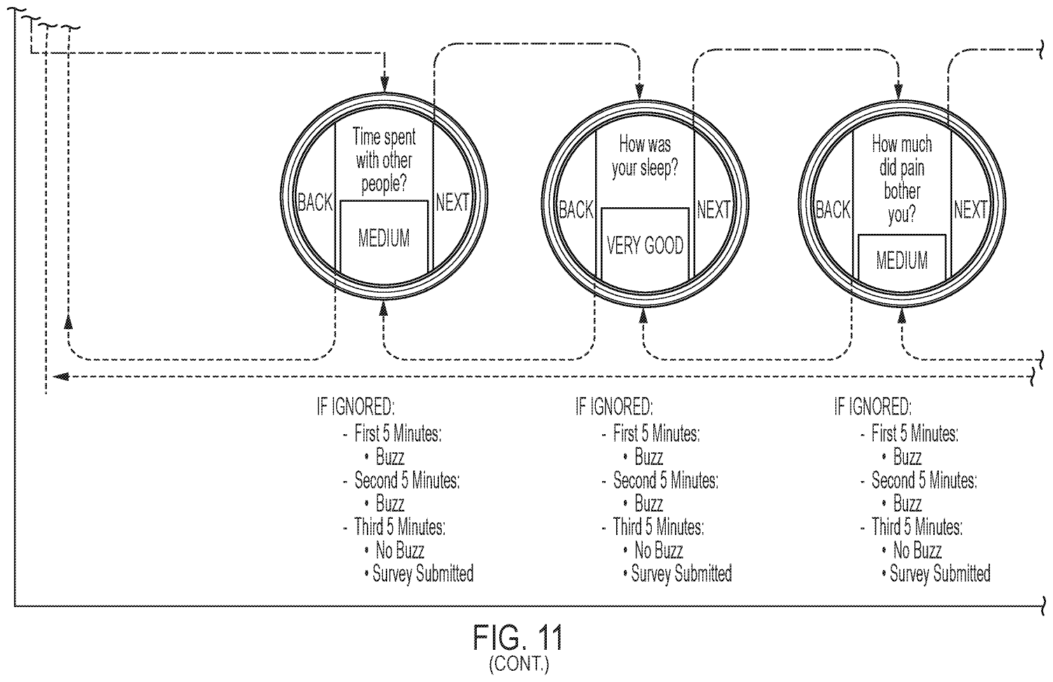

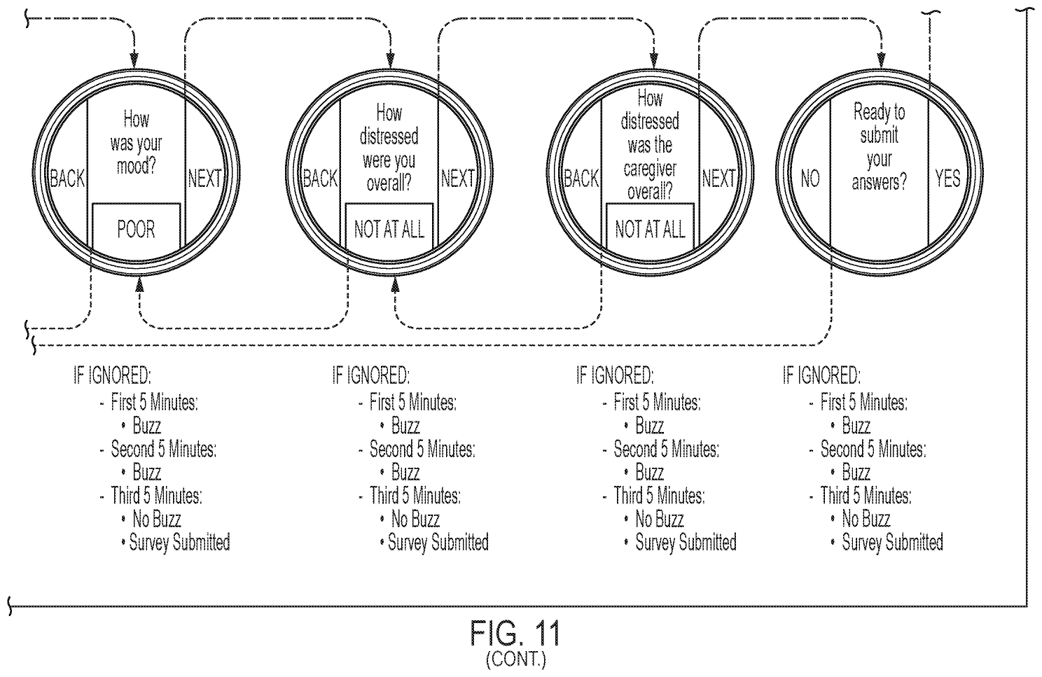

Turning to, FIG. 11, FIG. 11 provides a screenshot of selected examples of “Patient Automatic End of Day EMA” pertaining to, for example but not limited thereto, a patient survey at the end of the day that assesses general patient well-being, contextual factors and behaviors, in accordance with certain example embodiments. In an embodiment, this survey automatically appears at 8:30 pm and is available until midnight.

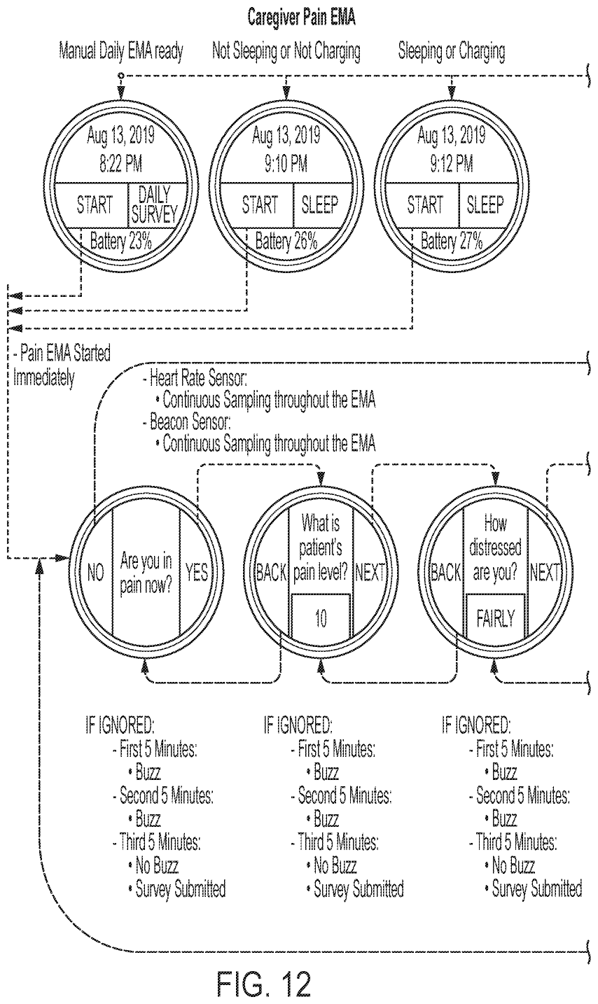

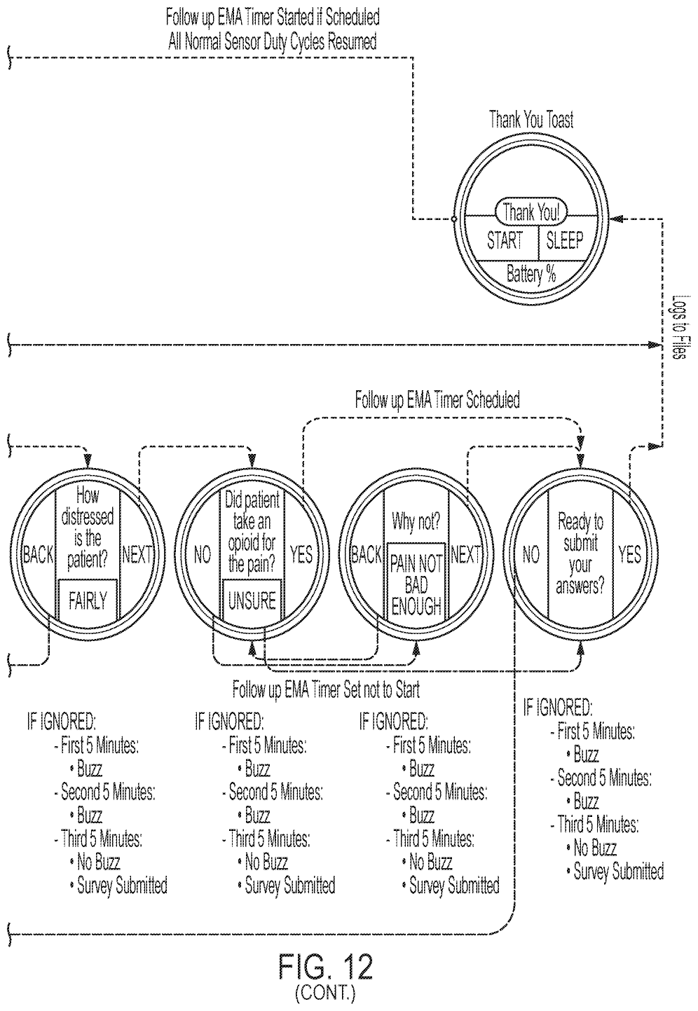

Turning to, FIG. 12, FIG. 12 provides a screenshot of selected examples of “Caregiver Pain EMA” pertaining to, for example but not limited thereto, how a caregiver initially marks and describes their perspective of a patient pain event, in accordance with certain example embodiments.

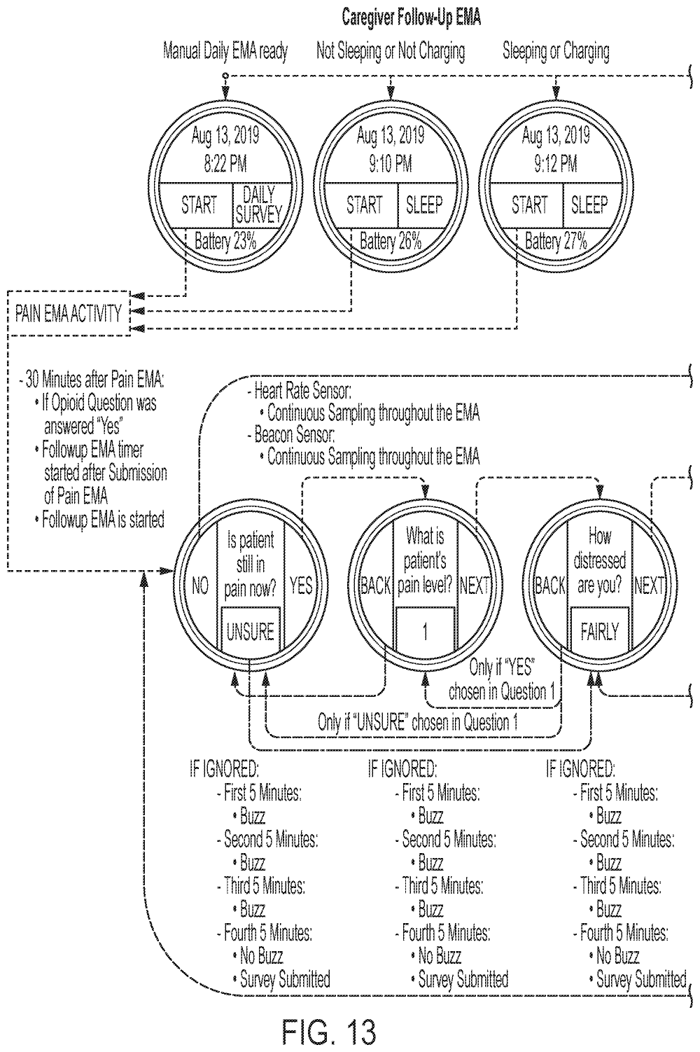

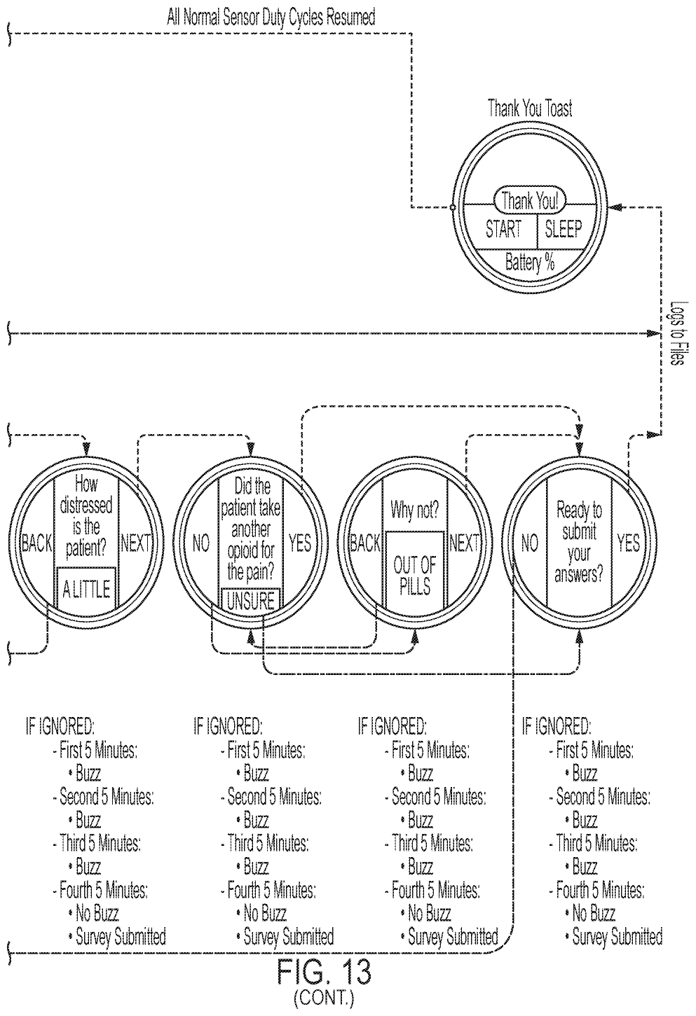

Turning to, FIG. 13, FIG. 13 provides a screenshot of selected examples of “Caregiver Follow-up EMA” pertaining to, for example but not limited thereto, how a caregiver describes pain 30 minutes after they report a patient uses a pharmacological or non-pharmacological strategy to reduce pain, in accordance with certain example embodiments.

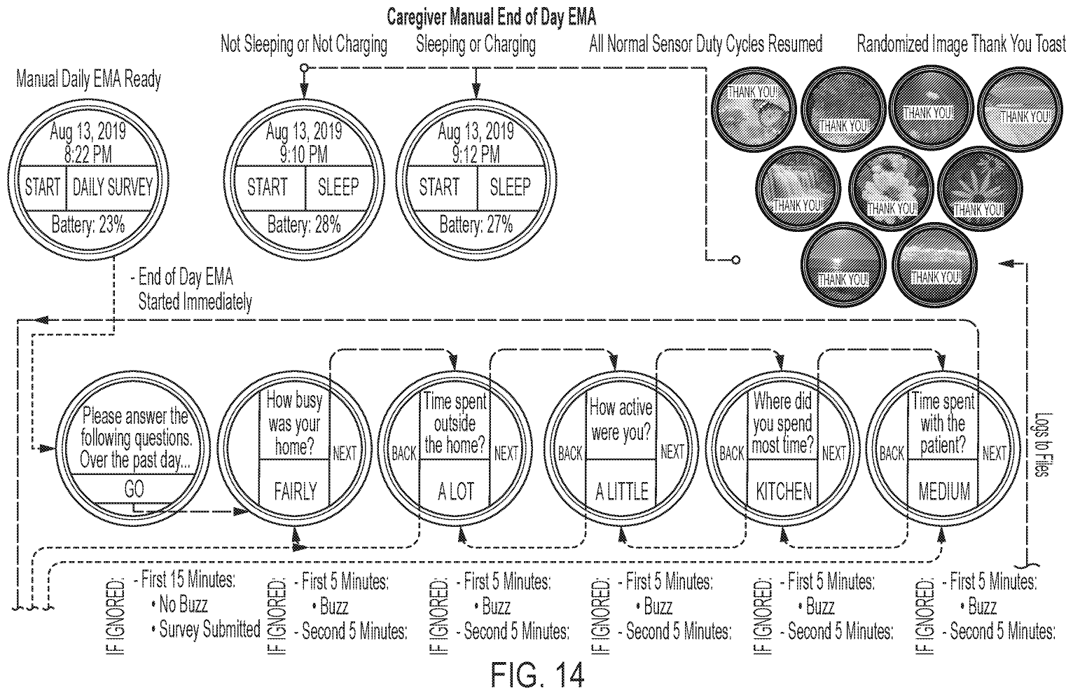

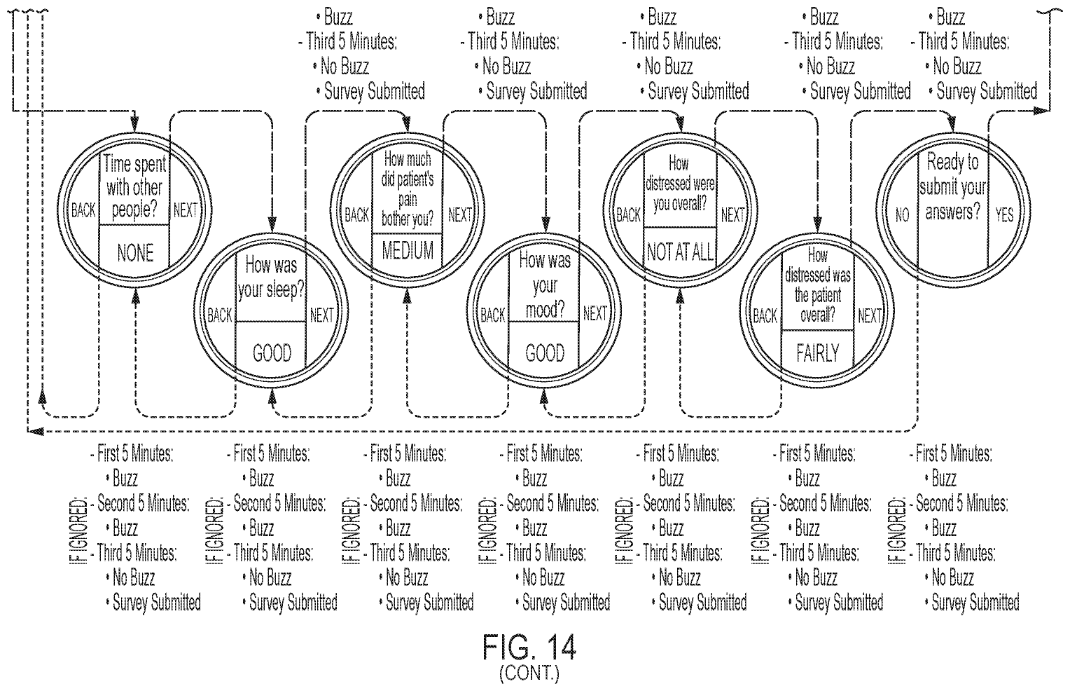

Turning to, FIG. 14, FIG. 14 provides a screenshot of selected examples of “Caregiver Manual End of Day EMA” pertaining to, for example but not limited thereto, a caregiver survey at the end of the day that assesses general caregiver well-being, contextual factors and behaviors, in accordance with certain example embodiments. In an embodiment, a caregiver manually generates this survey at time of their choice after 5 pm.

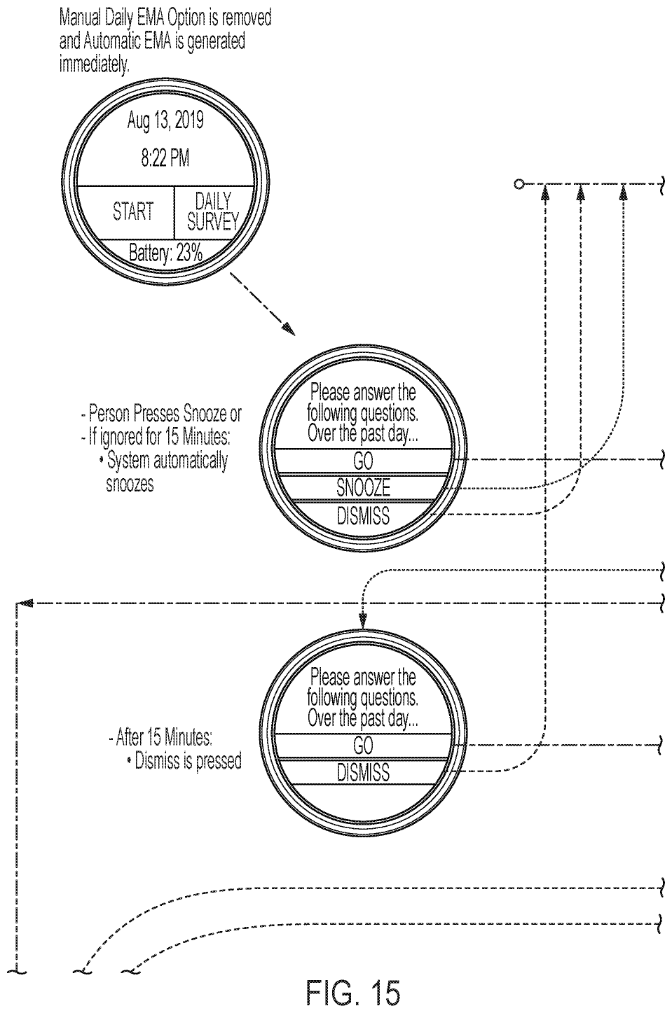

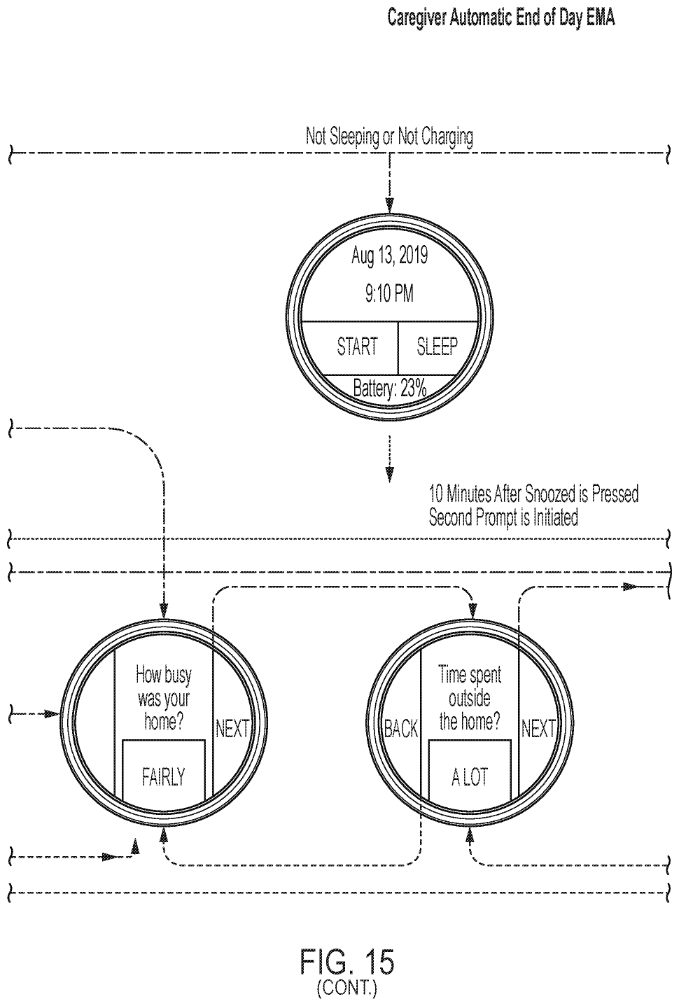

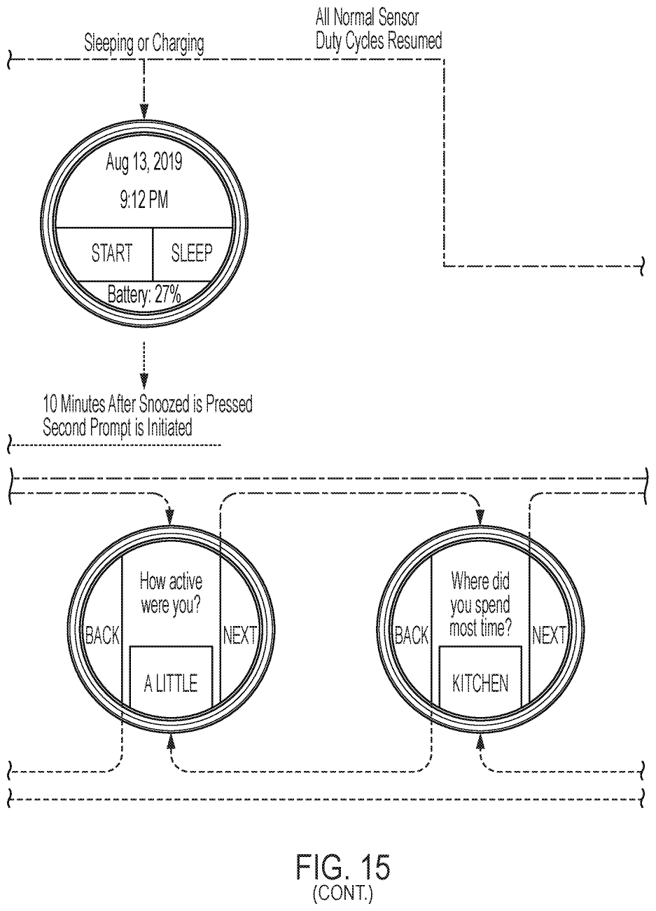

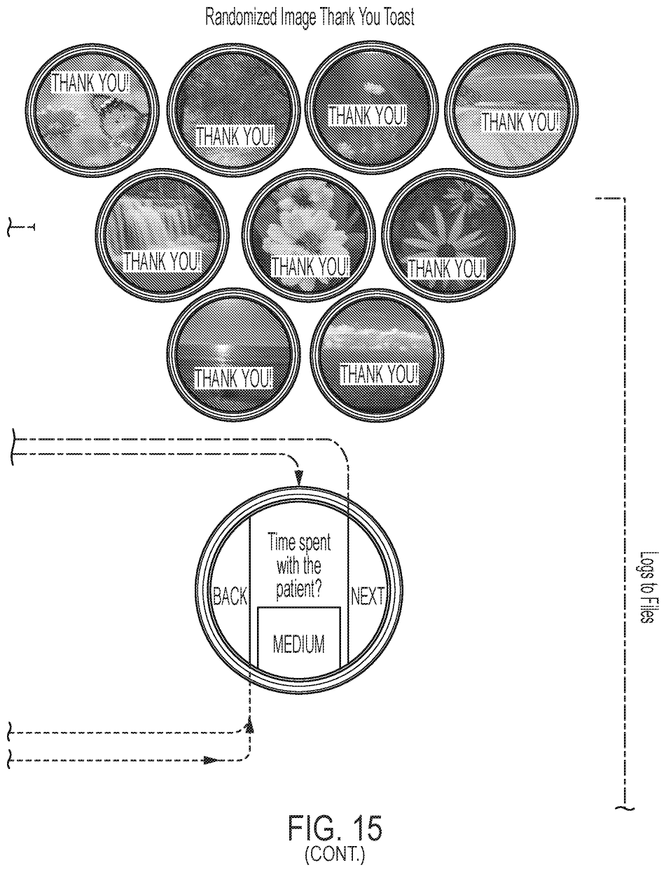

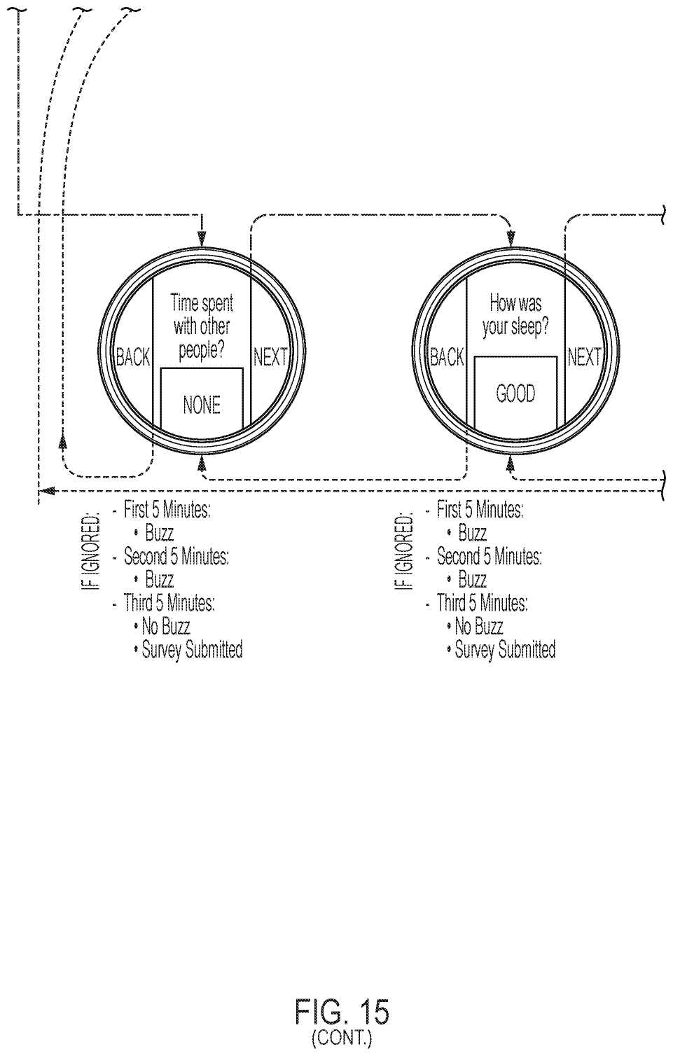

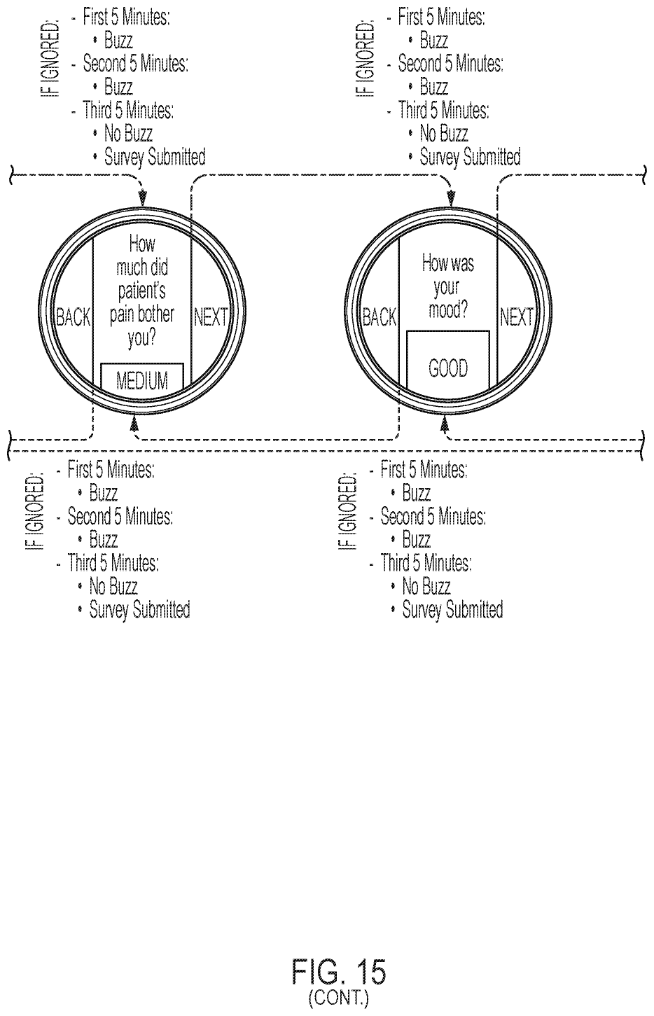

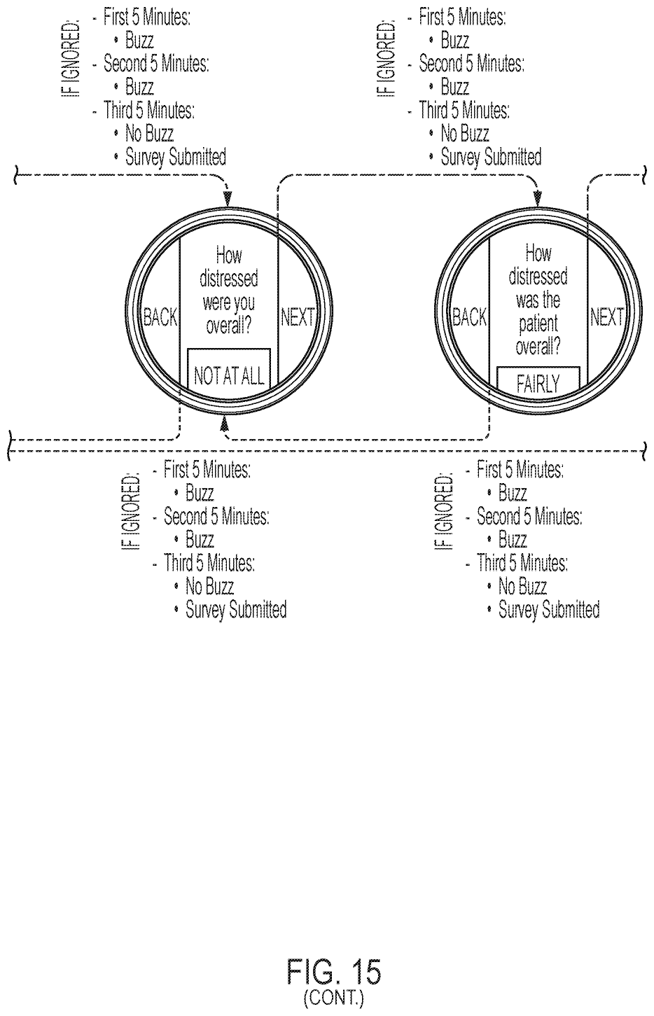

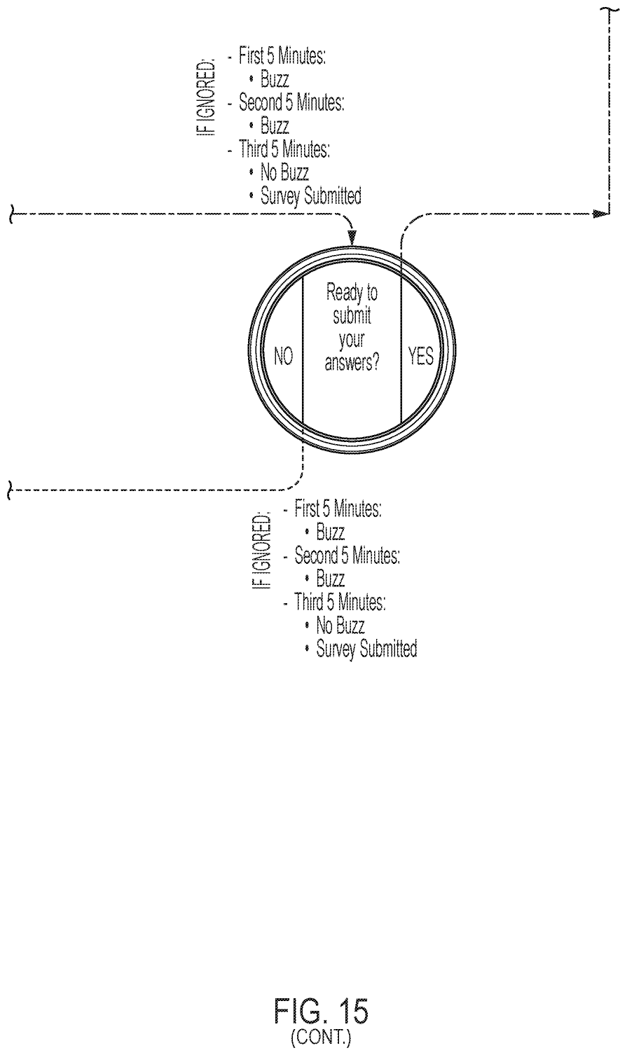

Turning to, FIG. 15, FIG. 15 provides a screenshot of selected examples of “Caregiver Automatic End of Day EMA” pertaining to, for example but not limited thereto, a caregiver survey at the end of the day that assesses general caregiver well-being, contextual factors and behaviors, in accordance with certain example embodiments. In an embodiment, this survey automatically appears at 8:30 pm and is available until midnight.

In an embodiment, the duty cycle process may entail a system sensor duty cycling that may include, but is not limited thereto, the following: pedometer sensor (step count), accelerometer sensor, photoplethysmography sensor (heart rate sensor), and localization sensor (beacon/estimote sensor). In an embodiment, the pedometer sensor may be always on, for example. In an embodiment, the accelerometer sensor may be sampling at about 5 Hz and disabled during sleep mode, for example. In an embodiment, the photoplethysmography sensor may be set for a five minute duty cycle (enabled for 30 seconds and disabled for 4 minutes and 30 seconds) and disabled during sleep mode, for example. In an embodiment, the localization sensor may be set for 1 minute and 30 second duty cycle (enabled for 15 seconds and disabled for 1 minute and 15 seconds) and disabled during sleep mode, for example.

In an embodiment, the process may entail, among other variations, the following settings: during the pain and follow-up EMA, the heart rate sensor and localization beacon are running continuously until the end of the survey (i.e., no duty cycling enabled); if no steps are detected after the localization sensor has run five times, then the sleep function is automatically enabled; and the sleep function disables all sensor and data gathering except for the pedometer sensor.

Expected Outcomes and Future Directions: Expected and realized outcomes from this research include: 1) knowledge about complex relationships among environmental, contextual, behavioral and physiological variables and pain events; 2) a process to create personalized, digital phenotypes related to advanced cancer pain; 3) a working prototype of data visualizations for future testing; and 4) identified specific pain and/or symptoms alleviating strategies based on predictors that can be modified/changed. Most importantly, we see one of the primary outcomes of this research as the ability to inform a variety of evidence-based personalized pain and symptom management interventions for both patients and/or caregivers that can be tested in clinical trials. For example, a next-step BESI-C clinical trial could deploy early notifications to modify the environment to prevent escalation of breakthrough pain and then evaluate this intervention on patient and caregiver pain and distress levels. We could also evaluate the impact of BESI-C pain management on patient/caregiver self-efficacy, caregiver burnout, and opioid-related adverse events. Looking further, we could test BESI-C impact on system-level factors, such as unplanned discharges from home hospice due to uncontrolled symptoms and explore how to integrate BESI-C data in to electronic health records.

In an embodiment, the method and system may be implemented using a “BESI Box” which is a contact-less deployment system (and related method). For example, instead of the implementer/administrator having to go to someone's home to set up the system the implementer/administrator would package it and mail to them (e.g., patient and caregiver at the home) with simple set up instructions; the patient and caregiver, for example, would collect the data and then send back the system to implementer/administrator for processing. Such embodiments may be particularly useful during any pandemic or so forth.

Final Remarks: This proposal provides for heterogeneous smart health sensing data—collected at the individual, dyad, and home levels, to characterize the complexity of advanced cancer pain in the home setting. Capturing these digital data will allow us to not only comprehensively describe the personalized and highly contextual nature of advanced cancer pain, from multiple perspectives, but also how to best generate and communicate collected data and discover predictors of breakthrough pain events. Perhaps most importantly, all aims of this study will inform the development of personalized, evidence-based strategies to reduce suffering and alleviate pain for both patients and family caregivers. In summary, the proposal is an excellent fit with various goals of an embodiment of the present invention, including, but not limited thereto: 1) Innovation and Technology; 2) Symptom Science; and 3) End of Life/Palliative Care.

### Additional Examples

Example 1. A computer-implemented method for monitoring and delivering in-situ real-time personalized intervention for a patient coping with cancer or non-cancer pain management or cancer-related or other disease-related symptoms by exchanging information with mobile devices and/or smartwatches in regards to a patient and caregiver dyad. The method may comprise:

collecting, by one or more computer devices associated with a health system, patient and caregiver dyadic in-situ data, wherein said patient and caregiver dyadic in-situ data is received from a patient user computing device and a caregiver user computing device;

wherein said patient user computing device and said caregiver user computing device associated with said health system are separate and distinct from said health system;

wherein said patient and caregiver dyadic in-situ data includes: environmental data, behavioral data, physiological data, and contextual data of each of a patient and caregiver;

receiving, by one or more computer devices associated with said health system, cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;

storing, by one or more computer devices associated with said health system, said cancer or non-cancer pain events data or cancer-related or other disease-related symptom events data of the patient;

relating, by one or more computer devices associated with said health system, said patient and caregiver dyadic in-situ data to said cancer or non-cancer pain events data or cancer-related or other disease-related symptom events data of patient;

generating, by one or more computer devices associated with said health system, real-time personalized intervention information for the patient and/or caregiver, based on said relation; and

communicating, by one or more computer devices associated with said health system, said real-time personalized intervention information, to said patient user computing device and caregiver user computing device for appropriate action to be undertaken, to anyone or more of the following: the caregiver, the patient, or both the caregiver and patient.

Example 2. The computer-implemented method of example 1, further comprising:

communicating, by one or more computer devices associated with said health system, said real-time personalized intervention information, to a participant user computing device for appropriate action to be undertaken.

Example 3. The computer-implemented method of example 2, wherein said participant user computing device is associated with a health care provider user or a clinician user.

Example 4. The computer-implemented method of example 1 (as well as subject matter of one or more of any combination of examples 2-3, in whole or in part), wherein said real-time personalized intervention comprises at least one or more of any combination of the following:

providing guidance of treatment for the patient and/or caregiver;

predicting occurrence of cancer or non-cancer pain events and/or magnitude of cancer or non-cancer pain events of patient; or

predicting cancer-related or other disease-related symptom events and/or magnitude of cancer related symptoms of patient.

Example 5. The computer-implemented method of example 4 (as well as subject matter of one or more of any combination of examples 2-3, in whole or in part), wherein said providing guidance of treatment for the patient and/or caregiver includes at least one or more of any combination of the following:

providing guidance regarding dosing and timing of medication for the patient;

providing guidance of pain management for the patient;

providing non-pharmacological treatment for the patient; or

providing behavioral, environmental or contextual modifications for the patient and/or caregiver.

Example 6. The computer-implemented method of example 1 (as well as subject matter of one or more of any combination of examples 2-5, in whole or in part), wherein at least one or more of said environmental data, behavioral data, physiological data, and contextual data are detected or sensed by an in-situ sensor or in-situ detector.

Example 7. The computer-implemented method of example 1 (as well as subject matter of one or more of any combination of examples 2-6, in whole or in part), wherein said environmental data includes ambient factors, in-situ, wherein in-situ defines a patient resident setting.

Example 8. The computer-implemented method of example 7 (as well as subject matter of one or more of any combination of examples 2-6, in whole or in part), wherein said ambient factor includes at least one or more of the following: temperature, light, noise, humidity or barometric pressure.

Example 9. The computer-implemented method of example 1 (as well as subject matter of one or more of any combination of examples 2-8, in whole or in part), wherein said behavioral data includes at least one or more of the following: ecological momentary assessment (EMA) data of patient and ecological momentary assessment (EMA) data of caregiver.

Example 10. The computer-implemented method of example 9 (as well as subject matter of one or more of any combination of examples 2-8, in whole or in part), wherein said EMA related behavioral data includes at least one or more of the following:

behavioral factors pertaining to what the patient or caregiver indicates as such actions that they do or actions that they take or report to take;

appetite of the patient and/or caregiver; or

energy level or fatigue level of the patient and/or caregiver.

Example 11. The computer-implemented method of example 9 (as well as subject matter in whole or in part of example 10), wherein said EMA related behavioral data includes at least one or more of the following:

pain medication use, reasons pain medication was not taken, or non-pharmacological strategies used to try to manage pain.

Example 12. The computer-implemented method of example 1 (as well as subject matter of one or more of any combination of examples 2-11, in whole or in part), wherein said physiological data includes at least one or more of the following: activity, movement, sleep, rest, or heart rate of the patient and caregiver.

Example 13. The computer-implemented method of example 1 (as well as subject matter of one or more of any combination of examples 2-12, in whole or in part), wherein said contextual data includes at least one or more of the following: ecological momentary assessment (EMA) data of patient or ecological momentary assessment (EMA) data of the caregiver.

Example 14. The computer-implemented method of example 13, wherein said EMA related contextual data includes at least one or more of the following:

factors pertaining to what is happening around the patient or caregiver or factors that may influence their experience;

appetite of the patient and/or caregiver; or energy level or fatigue level of the patient and/or caregiver.

Example 15. The computer-implemented method of example 13 (as well as subject matter in whole or in part of example 14), wherein:

in-situ defines a patient resident setting; and

said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much time was spent with the other member of the dyad; time spent with other people; overall pain interference; or overall distress levels.

Example 16. The computer-implemented method of example 1 (as well as subject matter of one or more of any combination of examples 2-15, in whole or in part), wherein in-situ being defined as a patient resident setting, and said contextual data includes at least one or more of the following:

location of the patient and caregiver within the patient resident setting; or

location of the patient and caregiver relative to one another, within the patient resident setting to define relative location.

Example 17. The computer-implemented method of example 16, wherein said contextual data further comprises:

said relative location of the patient and caregiver when a pain event occurs.

Example 18. A computer program product, wherein the computer program may comprise:

a non-transitory computer readable storage device having computer-executable program instructions embodied thereon that when executed by a computer processes information from mobile devices and/or smartwatches for monitoring and delivering in-situ real-time personalized intervention for a patient coping with cancer or non-cancer pain management or cancer-related or other disease-related symptoms in regards to a patient and caregiver dyad. In an embodiment, the computer-executable program instructions may comprise:
computer-executable program instructions to collect patient and caregiver dyadic in-situ data, wherein said patient and caregiver dyadic in-situ data is received from a patient user computing device and a caregiver user computing device, wherein said patient user computing device and said caregiver user computing device are separate and distinct from said computer;
wherein said patient and caregiver dyadic in-situ data includes: environmental data, behavioral data, physiological data, and contextual data of each of a patient and caregiver;
computer-executable program instructions to receive cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;
computer-executable program instructions to store said cancer or non-cancer pain events data or cancer-related or other disease-related symptom events data of the patient;
computer-executable program instructions to relate said patient and caregiver dyadic in-situ data to said cancer or non-cancer pain events data or cancer-related or other disease-related symptom events data of patient;
computer-executable program instructions to generate real-time personalized intervention information of the patient and/or caregiver, based on said relation; and
computer-executable program instructions to communicate said real-time personalized intervention information, to said patient user computing device and said caregiver user computing device for appropriate action to be undertaken, to anyone or more of the following: the caregiver, the patient, or both the caregiver and patient.

Example 19. The computer program product of example 18, further comprising:

computer-executable program instructions to communicate said real-time personalized intervention information, to a participant user computing device for appropriate action to be undertaken.

Example 20. The computer program product of example 19, wherein said participant user computing device is associated with a health care provider user or a clinician user.

Example 21. The computer program product of example 18 (as well as subject matter of one or more of any combination of examples 19-20, in whole or in part), wherein said real-time personalized intervention comprises at least one or more of any combination of the following:

providing guidance of treatment for the patient and/or caregiver;

predicting occurrence of cancer or non-cancer pain events and/or magnitude of cancer or non-cancer pain events of patient; or

predicting cancer-related symptom events and/or magnitude of cancer related symptoms of patient.

Example 22. The computer program product of example 21 (as well as subject matter of one or more of any combination of examples 19-20, in whole or in part), wherein said providing guidance of treatment for the patient and/or caregiver includes at least one or more of any combination of the following:

providing guidance regarding dosing and timing of medication for the patient;

providing guidance of pain management for the patient;

providing non-pharmacological treatment for the patient; or

providing behavioral, environmental or contextual modifications for the patient and/or caregiver.

Example 23. The computer program product of example 18 (as well as subject matter of one or more of any combination of examples 19-22, in whole or in part), wherein at least one or more of said environmental data, behavioral data, physiological data, and contextual data are detected or sensed by an in-situ sensor or in-situ detector.

Example 24. The computer program product of example 18 (as well as subject matter of one or more of any combination of examples 19-23, in whole or in part), wherein said environmental data includes ambient factors, in-situ, wherein in-situ defines a patient resident setting.

Example 25. The computer program product of example 24, wherein said ambient factor includes at least one or more of the following: temperature, light, noise, humidity or barometric pressure.

Example 26. The computer program product of example 18 (as well as subject matter of one or more of any combination of examples 19-25, in whole or in part), wherein said behavioral data includes at least one or more of the following: ecological momentary assessment (EMA) data of patient and ecological momentary assessment (EMA) data of caregiver.

Example 27. The computer program product of example 26, wherein said EMA related behavioral data includes behavioral at least one or more of the following:

factors pertaining to what the patient or caregiver indicates as such actions that they do or actions that they take or report to take;

appetite of the patient and/or caregiver; or energy level or fatigue of the patient and/or caregiver.

Example 28. The computer program product of example 26 (as well as subject matter in whole or in part of example 27), wherein said EMA related behavioral data includes at least one or more of the following:

pain medication use, reasons pain medication was not taken, or non-pharmacological strategies used to try to manage pain.

Example 29. The computer program product of example 18 (as well as subject matter of one or more of any combination of examples 19-28, in whole or in part), wherein said physiological data includes at least one or more of the following: activity, movement, sleep, rest, or heart rate of the patient and caregiver.

Example 30. The computer program product of example 18 (as well as subject matter of one or more of any combination of examples 19-29, in whole or in part), wherein said contextual data includes at least one or more of the following: ecological momentary assessment (EMA) data of patient or ecological momentary assessment (EMA) data of the caregiver.

Example 31. The computer program product of example 30, wherein said EMA related contextual data includes at least one or more of the following:

factors pertaining to what is happening around the patient or caregiver or factors that may influence their experience;

appetite of the patient and/or caregiver; or energy level or fatigue level of the patient and/or caregiver.

Example 32. The computer program product of example 30 (as well as subject matter in whole or in part of example 31), wherein:

in-situ defines a patient resident setting; and

said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much was time spent with the other member of the dyad; time spent with other people; overall pain interference; or overall distress levels.

Example 33 The computer program product of example 18 (as well as subject matter of one or more of any combination of examples 19-32, in whole or in part), wherein in-situ defines a patient resident setting, and said contextual data includes at least one or more of the following:

location of the patient and caregiver within the patient resident setting; or

location of the patient and caregiver relative to one another, within the patient resident setting to define relative location.

Example 34. The computer program product of example 33, wherein said contextual data further comprises:

said relative location of the patient and caregiver when a pain event occurs.

Example 35. A system to monitor and deliver in-situ real-time personalized intervention to mobile devices and/or smartwatches for a patient coping with cancer or non-cancer pain management or cancer-related or other disease-related symptoms in regards to a patient and caregiver dyad. In an embodiment, the system may comprise:

a storage resource;

a network module; and

a processor communicatively coupled to the storage resource and the network module, wherein the processor executes application code instructions that are stored in the storage resource and that cause the system to:
collect patient and caregiver dyadic in-situ data for a health system, wherein said patient and caregiver dyadic in-situ data is received from a patient user computing device and a caregiver user computing device;
wherein said patient user computing device and said caregiver user computing device associated with said health system are separate and distinct from said processor;
wherein said patient and caregiver dyadic in-situ data includes: environmental data, behavioral data, physiological data, and contextual data of each of a patient and caregiver;
receive cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;
store said cancer or non-cancer pain events data or cancer-related or other disease-related symptom events data of the patient;
relate said patient and caregiver dyadic in-situ data to said cancer or non-cancer pain events data or cancer-related or other disease-related symptom events data of patient;
generate real-time personalized intervention information of the patient and/or caregiver, based on said relation; and
communicate said real-time personalized intervention information, to said patient user computing device and said caregiver user computing device for appropriate action to be undertaken, to anyone or more of the following: the caregiver, the patient, or both the caregiver and patient.

Example 36. The system of example 35, wherein the processor is further configured to execute application code instructions that are stored in the storage resource and that cause the system to:
communicate said real-time personalized intervention information, to a participant user computing device for appropriate action to be undertaken.

Example 37. The system of example 36, wherein said participant user computing device is associated with a health care provider user or a clinician user.

Example 38. The system of example 35 (as well as subject matter of one or more of any combination of examples 36-37, in whole or in part), wherein said real-time personalized intervention comprises at least one or more of any combination of the following:

providing guidance of treatment for the patient and/or caregiver;

predicting occurrence of cancer or non-cancer pain events and/or magnitude of cancer or non-cancer pain events of patient; or

predicting cancer-related or other disease-related symptom events and/or magnitude of cancer related or other disease-related symptoms of patient.

Example 39. The system of example 38 (as well as subject matter of one or more of any combination of examples 36-37, in whole or in part), wherein said providing guidance of treatment for the patient and/or caregiver includes at least one or more of any combination of the following:

providing guidance regarding dosing and timing of medication for the patient;

providing guidance of pain management for the patient;

providing non-pharmacological treatment for the patient; or

providing behavioral, environmental or contextual modifications for the patient and/or caregiver.

Example 40. The system of example 35 (as well as subject matter of one or more of any combination of examples 36-39, in whole or in part), wherein at least one or more of said environmental data, behavioral data, physiological data, and contextual data are detected or sensed by an in-situ sensor or in-situ detector.

Example 41. The system of example 35 (as well as subject matter of one or more of any combination of examples 36-40, in whole or in part), wherein said environmental data includes ambient factors, in-situ, wherein in-situ defines a patient resident setting.

Example 42. The system of example 41, wherein said ambient factor includes at least one or more of the following: temperature, light, noise, humidity or barometric pressure.

Example 43. The system of example 35 (as well as subject matter of one or more of any combination of examples 36-42, in whole or in part), wherein said behavioral data includes at least one or more of the following: ecological momentary assessment (EMA) data of patient and ecological momentary assessment (EMA) data of caregiver.

Example 44. The system of example 43, wherein said EMA related behavioral data includes at least one or more of the following:

behavioral factors pertaining to what the patient or caregiver indicates as such actions that they do or actions that they take or report to take;

appetite of the patient and/or caregiver; or

energy level or fatigue level of the patient and/or caregiver.

Example 45. The system of example 43 (as well as subject matter in whole or in part of example 44), wherein said EMA related behavioral data includes at least one or more of the following:

pain medication use, reasons pain medication was not taken, or non-pharmacological strategies used to try to manage pain.

Example 46. The system of example 35 (as well as subject matter of one or more of any combination of examples 36-45, in whole or in part), wherein said physiological data includes at least one or more of the following: activity, movement, sleep, rest, or heart rate of the patient and caregiver.

Example 47. The system of example 35 (as well as subject matter of one or more of any combination of examples 36-46, in whole or in part), wherein said contextual data includes at least one or more of the following: ecological momentary assessment (EMA) data of patient or ecological momentary assessment (EMA) data of the caregiver.

Example 48. The system of example 47, wherein said EMA related contextual data includes at least one or more of the following:

factors pertaining to what is happening around the patient or caregiver or factors that may influence their experience;

appetite of the patient and/or caregiver; or

energy level or fatigue level of the patient and/or caregiver.

Example 49. The system of example 47 (as well as subject matter in whole or in part of example 48), wherein:

in-situ defines a patient resident setting; and

said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much time was spent with the other member of the dyad; time spent with other people; overall pain interference; or overall distress levels.

Example 50. The system of example 35 (as well as subject matter of one or more of any combination of examples 36-49, in whole or in part), wherein in-situ being defined as a patient resident setting, and said contextual data includes at least one or more of the following:

location of the patient and caregiver within the patient resident setting; or

location of the patient and caregiver relative to one another, within the patient resident setting to define relative location.

Example 51. The system of example 50, wherein said contextual data further comprises:

said relative location of the patient and caregiver when a pain event occurs.

### REFERENCES

The sensors, detectors, mobile devices, personal digital assistants (PDAs), wearable devices, smartwatches, smartphones, devices, systems, apparatuses, compositions, computer program products, non-transitory computer readable medium, networks, acquisition devices, and methods of various embodiments of the invention disclosed herein may utilize aspects (such as sensors, detectors, mobile devices, personal digital assistants (PDAs), wearable devices, smartwatches, smartphones, devices, apparatuses, systems, compositions, computer program products, non-transitory computer readable medium, networks, acquisition devices, and methods) disclosed in the following references, applications, publications and patents and which are hereby incorporated by reference herein in their entirety (and which are not admitted to be prior art with respect to the present invention by inclusion in this section):
1. U.S. Pat. No. 10,521,557 B2, Jain, et al., “Systems and Methods for Providing Dynamic, Individualized Digital Therapeutics for Cancer Prevention, Detection, Treatment, and Survivorship”, Dec. 31, 2019.
2. U.S. Patent Application Publication No. US 2016/0048659 A1, Pereira, et al, “Method and Tools for Predicting a Pain Response in a Subject Suffering from Cancer-Induced Bone Pain”, Feb. 18, 2016.
3. U.S. Pat. No. 10,496,788 B2, Amarasingham, et al., “Holistic Hospital Patient Care and Management System and Method for Automated Patient Monitoring”, Dec. 3, 2019.
4. International Patent Application Publication No. WO 2017/192784 A1, Chiang, “On-Demand All-Points Telemedicine Consultation System and Method”, Nov. 9, 2017.
5. U.S. Patent Application Publication No. US 2014/0089001 A1, Macoviak, et al., “Remotely-Executed Medical Diagnosis and Therapy Including Emergency Automation”, Mar. 27, 2014.
6. U.S. Pat. No. 9,798,860 B1, Movva, “Methods and Systems for Remotely Determining Levels of Healthcare Interventions”, Oct. 24, 2017.
7. U.S. Patent Application Publication No. US 2018/0103859 A1, Provenzano, “Systems, Devices, and/or Methods for Managing Patient Monitoring”, Apr. 19, 2018.
8. U.S. Pat. No. 8,554,195 B2, Rao, “Health Management System for Group Interactions Between Patients and Healthcare Practitioners”, Oct. 8, 2013.
9. U.S. Patent Application Publication No. US 2015/0223705 A1, Sadhu, “Multi-Functional User Wearable Portable Device”, Aug. 13, 2015.
10. U.S. Pat. No. 10,478,127 B2, Sampson, “Apparatuses, Methods, Processes, and Systems Related to Significant Detrimental Changes in Health Parameters and Activating Lifesaving Measures”, Nov. 19, 2019.
11. U.S. Pat. No. 8,521,563 B2, Severin, “Systems and Methods for Managing At-Home Medical Prevention, Recovery, and Maintenance”, Aug. 27, 2013.
12. U.S. Pat. No. 8,684,922 B2, Tran, “Health Monitoring System”, Apr. 1, 2014.
13. U.S. Pat. No. 10,610,111 B1, Tran, “Smart Watch”, Apr. 7, 2020.
14. U.S. Patent Application Publication No. US 2019/0147721 A1, Avitan, et al., “Personal Emergency Response System and Method for Improved Signal Initiation, Transmission, Notification/Annunciation, and Level of Performance”, May 16, 2019.
15. U.S. Patent Application Publication No. US 2015/0359489 A1, Baudenbacher, et al., “Smart Mobile Health Monitoring System and Related Methods”, Dec. 17, 2015.
16. International Patent Application Publication No. WO 2015/082555 A1, Oleynik, “Computational Medical Treatment Plan Method and System with Mass Medical Analysis”, Jun. 11, 2015.
17. International Patent Application Publication No. WO 2017/075496 A1, Tee, “A System and Method for Mobile Platform Designed for Digital Health Management and Support for Remote Patient Monitoring”, May 4, 2017.
18. U.S. Patent Application Publication No. US 2017/0124276 A1, Tee, “System and Method for Mobile Platform Designed for Digital Health Management and Support for Remote Patient Monitoring”, May 4, 2017.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A computer-implemented method for monitoring and delivering in-situ real-time personalized intervention for a patient coping with cancer or non-cancer pain management or cancer-related or other disease-related symptoms by exchanging information with mobile devices and/or smartwatches in regards to a patient and caregiver dyad, said method comprising:
collecting, by one or more computer devices associated with a health system, patient and caregiver dyadic in-situ data, wherein said patient and caregiver dyadic in-situ data is received from a patient user computing device and a caregiver user computing device;
wherein said patient user computing device and said caregiver user computing device associated with said health system are separate and distinct from said health system;
wherein said patient and caregiver dyadic in-situ data includes: environmental data, behavioral data, physiological data, and contextual data of each of a patient and caregiver;
receiving, by one or more computer devices associated with said health system, cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;
storing, by one or more computer devices associated with said health system, said cancer or non-cancer pain events data or cancer-related or other disease-related symptom events data of the patient;
relating, by one or more computer devices associated with said health system, said patient and caregiver dyadic in-situ data to said cancer or non-cancer pain events data or cancer-related or other disease-related symptom events data of patient;
generating, by one or more computer devices associated with said health system, real-time personalized intervention information for the patient and/or caregiver, based on said relation; and
communicating, by one or more computer devices associated with said health system, said real-time personalized intervention information, to said patient user computing device and caregiver user computing device for appropriate action to be undertaken, to anyone or more of the following: the caregiver, the patient, or both the caregiver and patient.

2. The computer-implemented method of claim 1, further comprising:
communicating, by one or more computer devices associated with said health system, said real-time personalized intervention information, to a participant user computing device for appropriate action to be undertaken.

3. The computer-implemented method of claim 2, wherein said participant user computing device is associated with a health care provider user or a clinician user.

4. The computer-implemented method of claim 1, wherein said real-time personalized intervention comprises at least one or more of any combination of the following:
providing guidance of treatment for the patient and/or caregiver;
predicting occurrence of cancer or non-cancer pain events and/or magnitude of cancer or non-cancer pain events of patient; or
predicting cancer-related or other disease-related symptom events and/or magnitude of cancer related symptoms of patient.

5. The computer-implemented method of claim 4, wherein said providing guidance of treatment for the patient and/or caregiver includes at least one or more of any combination of the following:
providing guidance regarding dosing and timing of medication for the patient;
providing guidance of pain management for the patient;
providing non-pharmacological treatment for the patient; or
providing behavioral, environmental or contextual modifications for the patient and/or caregiver.

6. The computer-implemented method of claim 1, wherein at least one or more of said environmental data, behavioral data, physiological data, and contextual data are detected or sensed by an in-situ sensor or in-situ detector.

7. The computer-implemented method of claim 1, wherein said environmental data includes ambient factors, in-situ, wherein in-situ defines a patient resident setting.

8. The computer-implemented method of claim 7, wherein said ambient factor includes at least one or more of the following: temperature, light, noise, humidity or barometric pressure.

9. The computer-implemented method of claim 1, wherein said behavioral data includes at least one or more of the following: ecological momentary assessment (EMA) data of patient and ecological momentary assessment (EMA) data of caregiver.

10. The computer-implemented method of claim 9, wherein said EMA related behavioral data includes at least one or more of the following:
behavioral factors pertaining to what the patient or caregiver indicates as such actions that they do or actions that they take or report to take;
appetite of the patient and/or caregiver; or
energy level or fatigue level of the patient and/or caregiver.

11. The computer-implemented method of claim 9, wherein said EMA related behavioral data includes at least one or more of the following:
pain medication use, reasons pain medication was not taken, or non-pharmacological strategies used to try to manage pain.

12. The computer-implemented method of claim 1, wherein said physiological data includes at least one or more of the following: activity, movement, sleep, rest, or heart rate of the patient and caregiver.

13. The computer-implemented method of claim 1, wherein said contextual data includes at least one or more of the following: ecological momentary assessment (EMA) data of patient or ecological momentary assessment (EMA) data of the caregiver.

14. The computer-implemented method of claim 13, wherein said EMA related contextual data includes at least one or more of the following:
factors pertaining to what is happening around the patient or caregiver or factors that may influence their experience;
appetite of the patient and/or caregiver; or
energy level or fatigue level of the patient and/or caregiver.

15. The computer-implemented method of claim 13, wherein:
in-situ defines a patient resident setting; and
said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much time was spent with the other member of the dyad; time spent with other people; overall pain interference; or overall distress levels.

16. The computer-implemented method of claim 1, wherein in-situ being defined as a patient resident setting, and said contextual data includes at least one or more of the following:
location of the patient and caregiver within the patient resident setting; or
location of the patient and caregiver relative to one another, within the patient resident setting to define relative location.

17. The computer-implemented method of claim 16, wherein said contextual data further comprises:
said relative location of the patient and caregiver when a pain event occurs.

18. A computer program product, comprising:
a non-transitory computer readable storage device having computer-executable program instructions embodied thereon that when executed by a computer processes information from mobile devices and/or smartwatches for monitoring and delivering in-situ real-time personalized intervention for a patient coping with cancer or non-cancer pain management or cancer-related or other disease-related symptoms in regards to a patient and caregiver dyad, said computer-executable program instructions comprising:
computer-executable program instructions to collect patient and caregiver dyadic in-situ data, wherein said patient and caregiver dyadic in-situ data is received from a patient user computing device and a caregiver user computing device, wherein said patient user computing device and said caregiver user computing device are separate and distinct from said computer;
wherein said patient and caregiver dyadic in-situ data includes: environmental data, behavioral data, physiological data, and contextual data of each of a patient and caregiver;
computer-executable program instructions to receive cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;
computer-executable program instructions to store said cancer or non-cancer pain events data or cancer-related or other disease-related symptom events data of the patient;
computer-executable program instructions to relate said patient and caregiver dyadic in-situ data to said cancer or non-cancer pain events data or cancer-related or other disease-related symptom events data of patient;
computer-executable program instructions to generate real-time personalized intervention information of the patient and/or caregiver, based on said relation; and
computer-executable program instructions to communicate said real-time personalized intervention information, to said patient user computing device and said caregiver user computing device for appropriate action to be undertaken, to anyone or more of the following: the caregiver, the patient, or both the caregiver and patient.

19. The computer program product of claim 18, further comprising:
computer-executable program instructions to communicate said real-time personalized intervention information, to a participant user computing device for appropriate action to be undertaken.

20. The computer program product of claim 19, wherein said participant user computing device is associated with a health care provider user or a clinician user.

21. The computer program product of claim 18, wherein said real-time personalized intervention comprises at least one or more of any combination of the following:
providing guidance of treatment for the patient and/or caregiver;
predicting occurrence of cancer or non-cancer pain events and/or magnitude of cancer or non-cancer pain events of patient; or
predicting cancer-related symptom events and/or magnitude of cancer related symptoms of patient.

22. The computer program product of claim 21, wherein said providing guidance of treatment for the patient and/or caregiver includes at least one or more of any combination of the following:
providing guidance regarding dosing and timing of medication for the patient;
providing guidance of pain management for the patient;
providing non-pharmacological treatment for the patient; or
providing behavioral, environmental or contextual modifications for the patient and/or caregiver.

23. The computer program product of claim 18, wherein at least one or more of said environmental data, behavioral data, physiological data, and contextual data are detected or sensed by an in-situ sensor or in-situ detector.

24. The computer program product of claim 18, wherein said environmental data includes ambient factors, in-situ, wherein in-situ defines a patient resident setting.

25. The computer program product of claim 24, wherein said ambient factor includes at least one or more of the following: temperature, light, noise, humidity or barometric pressure.

26. The computer program product of claim 18, wherein said behavioral data includes at least one or more of the following: ecological momentary assessment (EMA) data of patient and ecological momentary assessment (EMA) data of caregiver.

27. The computer program product of claim 26, wherein said EMA related behavioral data includes behavioral at least one or more of the following:
factors pertaining to what the patient or caregiver indicates as such actions that they do or actions that they take or report to take;
appetite of the patient and/or caregiver; or
energy level or fatigue of the patient and/or caregiver.

28. The computer program product of claim 26, wherein said EMA related behavioral data includes at least one or more of the following:
pain medication use, reasons pain medication was not taken, or non-pharmacological strategies used to try to manage pain.

29. The computer program product of claim 18, wherein said physiological data includes at least one or more of the following: activity, movement, sleep, rest, or heart rate of the patient and caregiver.

30. The computer program product of claim 18, wherein said contextual data includes at least one or more of the following: ecological momentary assessment (EMA) data of patient or ecological momentary assessment (EMA) data of the caregiver.

31. The computer program product of claim 30, wherein said EMA related contextual data includes at least one or more of the following:
factors pertaining to what is happening around the patient or caregiver or factors that may influence their experience;
appetite of the patient and/or caregiver; or
energy level or fatigue level of the patient and/or caregiver.

32. The computer program product of claim 30, wherein:
in-situ defines a patient resident setting; and
said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much was time spent with the other member of the dyad; time spent with other people; overall pain interference; or overall distress levels.

33. The computer program product of claim 18, wherein in-situ defines a patient resident setting, and said contextual data includes at least one or more of the following:
location of the patient and caregiver within the patient resident setting; or
location of the patient and caregiver relative to one another, within the patient resident setting to define relative location.

34. The computer program product of claim 33, wherein said contextual data further comprises:
said relative location of the patient and caregiver when a pain event occurs.

35. A system to monitor and deliver in-situ real-time personalized intervention to mobile devices and/or smartwatches for a patient coping with cancer or non-cancer pain management or cancer-related or other disease-related symptoms in regards to a patient and caregiver dyad, said system comprising:
a storage resource;
a network module; and
a processor communicatively coupled to the storage resource and the network module, wherein the processor executes application code instructions that are stored in the storage resource and that cause the system to:
collect patient and caregiver dyadic in-situ data for a health system, wherein said patient and caregiver dyadic in-situ data is received from a patient user computing device and a caregiver user computing device;
wherein said patient user computing device and said caregiver user computing device associated with said health system are separate and distinct from said processor;
wherein said patient and caregiver dyadic in-situ data includes: environmental data, behavioral data, physiological data, and contextual data of each of a patient and caregiver;
receive cancer or non-cancer pain events data or cancer-related or other disease-related symptom events data of the patient based on cancer or non-cancer pain events data or cancer-related or other disease-related symptom events data of the patient collected from said patient user computing device and/or said caregiver user computing device;
store said cancer or non-cancer pain events data or cancer-related or other disease-related symptom events data of the patient;
relate said patient and caregiver dyadic in-situ data to said cancer or non-cancer pain events data or cancer-related or other disease-related symptom events data of patient;
generate real-time personalized intervention information of the patient and/or caregiver, based on said relation; and
communicate said real-time personalized intervention information, to said patient user computing device and said caregiver user computing device for appropriate action to be undertaken, to anyone or more of the following: the caregiver, the patient, or both the caregiver and patient.

36. The system of claim 35, wherein the processor is further configured to execute application code instructions that are stored in the storage resource and that cause the system to:
communicate said real-time personalized intervention information, to a participant user computing device for appropriate action to be undertaken.

37. The system of claim 36, wherein said participant user computing device is associated with a health care provider user or a clinician user.

38. The system of claim 35, wherein said real-time personalized intervention comprises at least one or more of any combination of the following:
providing guidance of treatment for the patient and/or caregiver;
predicting occurrence of cancer or non-cancer pain events and/or magnitude of cancer or non-cancer pain events of patient; or
predicting cancer-related or other disease-related symptom events and/or magnitude of cancer related or other disease-related symptoms of patient.

39. The system of claim 38, wherein said providing guidance of treatment for the patient and/or caregiver includes at least one or more of any combination of the following:
providing guidance regarding dosing and timing of medication for the patient;
providing guidance of pain management for the patient;
providing non-pharmacological treatment for the patient; or
providing behavioral, environmental or contextual modifications for the patient and/or caregiver.

40. The system of claim 35, wherein at least one or more of said environmental data, behavioral data, physiological data, and contextual data are detected or sensed by an in-situ sensor or in-situ detector.

41. The system of claim 35, wherein said environmental data includes ambient factors, in-situ, wherein in-situ defines a patient resident setting.

42. The system of claim 41, wherein said ambient factor includes at least one or more of the following: temperature, light, noise, humidity or barometric pressure.

43. The system of claim 35, wherein said behavioral data includes at least one or more of the following: ecological momentary assessment (EMA) data of patient and ecological momentary assessment (EMA) data of caregiver.

44. The system of claim 43, wherein said EMA related behavioral data includes at least one or more of the following:
behavioral factors pertaining to what the patient or caregiver indicates as such actions that they do or actions that they take or report to take;
appetite of the patient and/or caregiver; or
energy level or fatigue level of the patient and/or caregiver.

45. The system of claim 43, wherein said EMA related behavioral data includes at least one or more of the following:
pain medication use, reasons pain medication was not taken, or non-pharmacological strategies used to try to manage pain.

46. The system of claim 35, wherein said physiological data includes at least one or more of the following: activity, movement, sleep, rest, or heart rate of the patient and caregiver.

47. The system of claim 35, wherein said contextual data includes at least one or more of the following: ecological momentary assessment (EMA) data of patient or ecological momentary assessment (EMA) data of the caregiver.

48. The system of claim 47, wherein said EMA related contextual data includes at least one or more of the following:
factors pertaining to what is happening around the patient or caregiver or factors that may influence their experience;
appetite of the patient and/or caregiver; or
energy level or fatigue level of the patient and/or caregiver.

49. The system of claim 47, wherein:
in-situ defines a patient resident setting; and
said EMA related contextual data includes at least one or more of the following:
pain severity; how busy/active was the patient resident setting; distress levels; sleep quality and quantity; mood; current location; time spent outside the patient resident setting; activity level; energy level; fatigue; appetite; room in the patient resident setting where they spent most time; how much time was spent with the other member of the dyad; time spent with other people; overall pain interference; or overall distress levels.

50. The system of claim 35, wherein in-situ being defined as a patient resident setting, and said contextual data includes at least one or more of the following:
location of the patient and caregiver within the patient resident setting; or
location of the patient and caregiver relative to one another, within the patient resident setting to define relative location.

51. The system of claim 50, wherein said contextual data further comprises:
said relative location of the patient and caregiver when a pain event occurs.
